(19) United States
(12) Patent Application Publication
(10) Pub. No.: US 2024/0033004 A1
(43) Pub. Date: Feb. 1, 2024

(54) MODULATING SURGICAL DEVICE SETTINGS BASED ON TISSUE WHITENING

(71) Applicant: GYRUS ACMI, INC. D/B/A OLYMPUS SURGICAL TECHNOLOGIES AMERICA, Westborough, MA (US)

(72) Inventors: Kurt G. Shelton, Bedford, MA (US); Kristen R. Toutant, Denver, CO (US)

(21) Appl. No.: 18/350,935

(22) Filed: Jul. 12, 2023

Related U.S. Application Data
(60) Provisional application No. 63/369,099, filed on Jul. 22, 2022.

(51) Int. Cl. A61B 18/26; A61B 1/00; A61B 5/103

(52) U.S. Cl. CPC A61B 18/26; A61B 1/00009; A61B 5/1032; A61B 2018/00791

(57) ABSTRACT

Systems, devices, and methods for automatic control of surgical site temperature during an endoscopic procedure are disclosed. An exemplary endoscopic surgical system comprises an endoscopic surgical device controllably coupled to a medical instrument to deliver energy to a surgical site during a procedure, an imaging sensor to generate images or video frames of at least a portion of the surgical site during the procedure, and a controller circuit to analyze the generated images or video frames to determine whether a degree of heat built up in a first target at the surgical site exceeds a predetermined threshold. Based on such determination, the controller circuit can determine whether to adjust at least one operating parameter associated with the endoscopic surgical system to achieve or maintain a treatment effect of a different second target at the surgical site while avoid damaging the first target during the procedure.

### PRIORITY CLAIM

This application claims the benefit of priority to U.S. Provisional Patent Application Ser. No. 63/369,099, filed Jul. 22, 2022, the contents of which are incorporated herein by reference.

### TECHNICAL FIELD

This invention relates generally to an endoscopic surgical system, and more specifically relates to approaches for modulating one or more settings of the endoscopic surgical system based on tissue whitening detected from images or video frames of at least a portion of the surgical site.

### BACKGROUND

Endoscopes are typically used to provide access to an internal location of a patient so that a doctor is provided with visual access. Some endoscopes are used in minimally invasive surgery to remove unwanted tissue or foreign objects from the body of the patient. For example, a nephroscope is used by a clinician to inspect the renal system, and to perform various procedures under direct visual control. In a percutaneous nephrolithotomy (PCNL) procedure, a nephroscope is placed through the patient's flank into the renal pelvis. Calculi or mass from various regions of a body including, for example, urinary system, gallbladder, nasal passages, gastrointestinal tract, stomach, or tonsils, can be visualized and extracted.

Various medical instruments such as laser or plasma systems have been used for delivering surgical laser energy to various target treatment areas such as soft or hard tissue. Examples of the laser treatment include ablation, coagulation, vaporization, fragmentation, etc. In lithotripsy applications, laser has been used to break down calculi structures in kidney, gallbladder, ureter, among other stone-forming regions, or to ablate large calculi into smaller fragments. The calculi fragments may be removed via a working channel of an endoscope (e.g., an ureteroscope) or may be passed naturally by the patient following the procedure.

Heat buildup is a potentially hazardous consequence of laser treatment of an anatomical or calculi target, particularly in cases where relatively high intensity laser output is used in the treatment, such as laser lithotripsy to ablate or fragment a calculi target of certain size, shape, hardness, or compositions. Excessive heat built up at or near the surgical site may cause thermal damage of non-target tissue or organs.

### SUMMARY

Effective surgical site temperature control can help prevent tissue thermal damage caused by heat buildup during medical procedures such as laser lithotripsy or ultrasound lithotripsy procedures. Conventionally, temperature is sensed from a surgical site and displayed to a user (e.g., a physician) during the procedure. The user can manually change settings of the medical instruments (e.g., laser output intensity, or temporarily turn off the laser, if the surgical site temperature reaches or exceeds a safety limit. Such manual temperature adjustment technique may not provide precise temperature control at the surgical site. Additionally, adjustment of the settings of the medical instruments (e.g., laser output intensity may not achieve adequate and fast temperature relief at the surgical site. For example, in some cases, reducing laser output intensity or shutting off laser output may compromise therapy efficiency and/or extend procedure time. It should be noted that the medical instrument herein refers to a laser system, but any suitable medical instruments, such as an ultrasound system, which may be coupled to or implemented in an endoscope for providing treatment or diagnosis of a target are also within the scope of the present invention.

The present invention describes systems, devices, and methods to improve surgical site temperature control by automatically adjusting one or more device settings based on tissue whitening (also referred to as tissue blanching) that can be detected from images or video frames of at least a portion of the surgical site. Tissue whitening can be an early indication of a risk of laser-induced tissue thermal damage. According to one embodiment, an exemplary endoscopic surgical system comprises an endoscopic surgical device controllably coupled to a medical instrument (e.g., a laser system) and configured to deliver energy (e.g., laser energy) to a surgical site during a procedure, an imaging sensor configured to generate images or video frames of at least a portion of the surgical site during the procedure, and a controller circuit configured to analyze the generated images or video frames to determine whether a degree of heat built up in a first target at the surgical site exceeds a predetermined threshold. The predetermined threshold, which can be different for different types of tissue, can be represented by a threshold temperature (e.g., 42° C.) that starts to cause undesirable clinical effects on the tissue. As there is a positive correlation between the degree of tissue whitening and the degree of heat buildup, the degree of heat buildup can be inferred by evaluating the degree tissue whitening in the first target, and the predetermined threshold for heat buildup can be represented by a threshold degree of tissue whitening. Based on such determination, the controller circuit can determine whether to adjust at least one operating parameter associated with the endoscopic surgical system so as to achieve or maintain a treatment effect of a second target at the surgical site different from the first target, while avoid damaging the first target during the procedure. The surgical site temperature control approach described herein may advantageously prevent, or reduce the severity of, tissue thermal damage induced by energy (e.g., laser energy) delivered to the tissue site. Various temperature control means allow for more versatile control of surgical site temperature in accordance with the surgical site conditions. The alternative temperature control means (e.g., irrigation or suction flow and irrigant treatment) can help avoid discontinuation or substantial reduction of energy output in an endoscopic procedure (e.g., a laser or ultrasound lithotripsy procedure). As such, more precise and faster temperature control and improved laser therapy efficacy and tissue safety can be achieved.

Example 1 is a endoscopic surgical system, comprising: an endoscopic surgical device, controllably coupled to a medical instrument for delivering energy to a surgical site during a procedure; an imaging sensor configured to generate images or video frames of at least a portion of the surgical site during the procedure, and a controller circuit configured to: analyze the generated images or video frames to determine whether a degree of heat built up in a first target at the surgical site exceeds a predetermined threshold; and based on the determination, determine whether to adjust at least one operating parameter associated with the endoscopic surgical system so as to achieve or maintain a treatment effect of a second target at the surgical site while avoid damaging the first target during the procedure, the second target being different from the first target.

In Example 2, the subject matter of Example 1 optionally includes, wherein the first target comprises tissue in a urinary system, the second target comprises a calculi target, and the medical instrument comprises at least one laser system for delivering laser energy to treat the calculi target at the surgical site.

In Example 3, the subject matter of any one or more of Examples 1-2 optionally include, wherein the controller circuit is further configured to: detect, in the images or video frames, a change in intensity of one or more color components over time associated with the first target in the images or video frames; and determine whether tissue whitening has occurred at the first target based on the detected change in intensity of the one or more color components.

In Example 4, the subject matter of any one or more of Examples 1-3 optionally include, wherein: the endoscopic surgical device is configured to direct an aiming beam from a light source to the surgical site, the aiming beam having a characteristic color component; and the controller circuit is further configured to identify a footprint of the aiming beam in the generated images or video frames, and determine the degree of heat built up in the first target based on an increase in intensity of the characteristic color component at a vicinity of the footprint of the aiming beam.

In Example 5, the subject matter of any one or more of Examples 1-4 optionally include, wherein the controller circuit is further configured to: determine a rate of heat built up in the first target based on a comparison of the images or video frames taken at different times during the procedure; and adjust the at least one operating parameter associated with the endoscopic surgical system in accordance with the determined degree or rate of tissue whitening.

In Example 6, the subject matter of Example 2 optionally includes, wherein the at least one operating parameter to be adjusted includes a laser output setting of the at least one laser system.

In Example 7, the subject matter of Example 6 optionally includes, wherein the laser output setting comprises at least one of: a pulse width of a laser pulse; a pulse shape of a laser pulse; a peak power of a laser pulse; or a pulse frequency representing a number of laser pulses per unit time.

In Example 8, the subject matter of Example 7 optionally includes, wherein the controller circuit is further configured to perform the adjustment of the laser output setting automatically upon determining that the degree of heat built up in the first target exceeds the predetermined threshold.

In Example 9, the subject matter of any one or more of Examples 6-8 optionally include, wherein the controller circuit is further configured to adjust the laser output setting so as to produce a non-equilibrium irrigation flow or to promote collapses of vaporized bubbles induced by the laser energy.

In Example 10, the subject matter of Example 9 optionally includes, wherein the laser output setting to be adjusted comprises a pulse sequencing representing a temporal distribution of laser pulses within a specific time interval, the laser pulses being delivered to the second target in accordance with the adjusted pulse sequencing.

In Example 11, the subject matter of Example 10 optionally includes, wherein to adjust the pulse sequencing, the controller circuit is further configured to randomize respective timings of the laser pulses within the specific time interval.

In Example 12, the subject matter of any one or more of Examples 6-11 optionally include, wherein to adjust the laser output setting, the controller circuit is further configured to prioritize adjustments of a pulse shape or a pulse sequencing over an adjustment of an average power of the laser pulses.

In Example 13, the subject matter of any one or more of Examples 1-12 optionally include an irrigation and/or suction system configured to provide irrigant into, and suction of fluid from, the surgical site.

In Example 14, the subject matter of Example 13 optionally includes, wherein the at least one operating parameter associated with the endoscopic surgical system comprises at least one of an irrigation flow or a suction flow associated with an irrigation system and a suction system, respectively.

In Example 15, the subject matter of Example 14 optionally includes a pressure sensor configured to sense a pressure at the surgical site during the procedure, wherein the controller circuit is further configured to selectively increase the irrigation flow or the suction flow via the irrigation and/or suction system, including to: increase the suction flow but not the irrigation flow when the sensed pressure exceeds an upper pressure limit; increase one or both of the irrigation flow or the suction flow when the sensed pressure is within a range defined by the upper pressure limit and a lower pressure limit; and increase the irrigation flow but not the suction flow when the sensed pressure falls below the lower pressure limit.

In Example 16, the subject matter of any one or more of Examples 13-15 optionally include an irrigant treatment unit configured to alter a temperature of the irrigant, wherein the controller circuit is further configured to generate a control signal to the irrigant treatment unit to adjust the temperature of the irrigant before reaching the surgical site upon determining that the degree of heat built up in the first target exceeds the predetermined threshold.

In Example 17, the subject matter of any one or more of Examples 2 and 6-12 optionally include, wherein the endoscopic surgical device includes an optical pathway with an adjustable distal portion, the optical pathway configured to direct the laser energy to the surgical site, wherein the controller circuit is further configured to, upon determining that the degree of heat built up in the first target exceeds the predetermined threshold, generate a control signal to an actuator coupled to the optical pathway to adjust a position or orientation of the distal portion of the optical pathway relative to the surgical site.

In Example 18, the subject matter of any one or more of Examples 1-17 optionally include, wherein the at least one operating parameter associated with the endoscopic surgical system comprises at least one of: temperature of an irrigant before being applied to the surgical site; an irrigation flow rate; a suction flow rate; or a laser output setting of a laser system.

In Example 19, the subject matter of Example 18 optionally includes, wherein the controller circuit is further configured to perform the adjustment with a bias toward one of the operating parameters based at least in part on at least one of the degree of heat built up in the first target or a pressure at the surgical site.

In Example 20, the subject matter of Example 19 optionally includes, wherein the controller circuit is further configured to, upon determining that the pressure at the surgical site is substantially below a maximal allowable pressure, adjust at least one of the irrigation flow rate or the suction flow rate prior to adjusting the laser output setting.

In Example 21, the subject matter of any one or more of Examples 19-20 optionally include, wherein the controller circuit is further configured to, upon determining that the pressure at the surgical site is substantially close to a maximal allowable pressure, adjust the laser output setting prior to adjusting the irrigation flow rate or the suction flow rate.

In Example 22, the subject matter of any one or more of Examples 1-21 optionally include a user interface device configured to generate an alert upon determining that the degree of heat built up in the first target exceeds the predetermined threshold.

In Example 23, the subject matter of any one or more of Examples 1-22 optionally include a user interface device, wherein the controller circuit is further configured to generate a recommended adjustment of the at least one operating parameter, and to receive a user input to confirm, reject, or modify the recommended adjustment.

Example 24 is a method for controlling temperature at a surgical site of a patient during an endoscopic procedure using an endoscopic surgical system, the method comprising: directing energy produced by a medical instrument to the surgical site; generating images or video frames of at least a portion of the surgical site using an imaging sensor; analyzing the generated images or video frames and determining whether a degree of heat built up in a first target at the surgical site exceeds a predetermined threshold; and based on the determination, determining whether to adjust at least one operating parameter associated with the endoscopic surgical system so as to achieve or maintain a treatment effect of a second target at the surgical site while avoid damaging the first target during the procedure, the second target being different from the first target.

In Example 25, the subject matter of Example 24 optionally includes, wherein the first target comprises tissue in a urinary system, the second target comprises a calculi target, and the energy produced by the medical instrument comprises laser energy produced by at least one laser system to treat the calculi target at the surgical site.

In Example 26, the subject matter of Example 25 optionally includes, wherein the at least one operating parameter to be adjusted includes a laser output setting of the at least one laser system, the laser output setting comprising at least one of a pulse width of a laser pulse; a pulse shape of a laser pulse; a peak power of a laser pulse; or a pulse frequency representing a number of laser pulses per unit time.

In Example 27, the subject matter of Example 26 optionally includes, wherein the laser output setting to be adjusted includes a pulse sequencing representing a temporal distribution of laser pulses within a specific time interval, the laser pulses being delivered to the surgical site in accordance with the adjusted pulse sequencing producing a non-equilibrium irrigation flow and promoting collapses of vaporized bubbles induced by the laser pulses.

In Example 28, the subject matter of Example 27 optionally includes, wherein adjusting the pulse sequencing includes randomizing respective timings of the laser pulses within the specific time interval.

In Example 29, the subject matter of any one or more of Examples 24-28 optionally include, detecting, in the images or video frames, a change in intensity of one or more color components over time associated with the first target in the images or video frames; and determining whether tissue whitening has occurred at the first target based at least in part on the detected change in intensity of the one or more color components.

In Example 30, the subject matter of any one or more of Examples 24-29 optionally include: directing an aiming beam from a light source to the surgical site, the aiming beam having a characteristic color component; identifying a footprint of the aiming beam in the generated images or video frames; and determine the degree of heat built up in the first target based at least in part on an increase in intensity of the characteristic color component at a vicinity of the footprint of the aiming beam.

In Example 31, the subject matter of any one or more of Examples 24-30 optionally include: determining a rate of heat built up in the first target based on a comparison of the images or video frames taken at different times during the procedure; and adjusting the at least one operating parameter associated with the endoscopic surgical system in accordance with the determined degree or rate of tissue whitening.

In Example 32, the subject matter of any one or more of Examples 24-31 optionally include, wherein adjusting the at least one operating parameter associated with the endoscopic surgical system includes adjusting at least one of an irrigation flow of irrigant into the surgical site or a suction flow of fluid out of the surgical site.

In Example 33, the subject matter of Example 32 optionally includes sensing a pressure at the surgical site during the procedure using a pressure sensor, wherein adjusting at least one of the irrigation flow or the suction flow includes: increasing the suction flow but not the irrigation flow when the sensed pressure exceeds an upper pressure limit; increasing one or both of the irrigation flow or the suction flow when the sensed pressure is within a range defined by the upper pressure limit and a lower pressure limit; and increasing the irrigation flow but not the suction flow when the sensed pressure falls below the lower pressure limit.

In Example 34, the subject matter of any one or more of Examples 24-33 optionally include, wherein adjusting the at least one operating parameter includes adjusting, via an irrigant treatment unit coupled to an irrigation and/or suction system, a temperature of the irrigant before reaching the surgical site upon determining that the degree of heat built up in the first target exceeds the predetermined threshold.

In Example 35, the subject matter of any one or more of Examples 24-34 optionally include, wherein adjusting the at least one operating parameter includes, upon determining that the degree of heat built up in the first target exceeds the predetermined threshold, adjusting a position or orientation of a distal portion of an optical pathway relative to the surgical site, and directing the energy to the surgical site via the optical pathway.

In Example 36, the subject matter of any one or more of Examples 24-35 optionally include prioritizing adjustments of two or more operating parameters of the endoscopic surgical system including: a temperature of an irrigant before being applied to the surgical site; an irrigation flow rate; a suction flow rate; and a laser output setting of a laser system.

In Example 37, the subject matter of any one or more of Examples 24-36 optionally include, upon determining that the degree of heat built up in the first target exceeds the predetermined threshold: generating an alert; or generating a recommended adjustment of the at least one operating parameter and receiving a user input to confirm, reject, or modify the recommended adjustment.

This summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 is a block diagram illustrating an example of a laser energy delivery system configured to provide laser treatment to an anatomical target at or near a surgical site.

FIG. 2 is a block diagram illustrating an endoscopic surgical system with automatic surgical site condition control, and at least a part of the environment in which the system may operate.

FIG. 3 illustrates an example of an endoscopic laser lithotripsy system with automatic surgical site condition control.

FIG. 4 illustrates an endoscopic image of the illuminated target site including an aiming beam footprint.

FIGS. SA-5D illustrate an example of changing the laser pulse sequencing and resultant irrigation flow and bubble state.

FIG. 6 is a flowchart illustrating an example method of controlling surgical site temperature during a laser procedure for treating an anatomical target.

FIG. 7A is a flowchart illustrating an example method of generating a temperature management plan based on surgical site conditions such as tissue whitening and/or pressure at the surgical site.

FIG. 7B is a flowchart illustrating an example of a temperature management plan with prioritized means for controlling surgical site temperature.

FIG. 8 is a block diagram illustrating an example machine upon which any one or more of the techniques (e.g., methodologies) discussed herein may perform.

### DETAILED DESCRIPTION

An endoscopic procedure is a medical procedure of viewing and operating on an internal organ, and/or delivering energy (e.g., laser energy or ultrasound energy) to a target body region to achieve a particular diagnostic or therapeutic effect. For example, laser endoscopy have been used for treatment of soft and hard tissue (e.g., damaging or destroying cancer cells), or in lithotripsy applications. During the procedure, a practitioner can insert a scope through an incision in a patient's ureter and into the patient's kidney. Through the scope, the practitioner can locate certain stones in the kidney or upper ureter, break the stones into smaller fragments by illuminating the stone, through the scope, with relatively high-powered infrared laser beam, be laser beam can ablate a stone into smaller fragments. The stone fragments can then be withdrawn from the kidney. The scope can include an endoscope, a nephroscope, and/or a cystoscope.

Laser energy delivered to the environment of at least a portion of the surgical site and laser treatment of anatomical target (e.g., ablation and fragmentation of a calculi target) may cause heat built up at or near the surgical site, particularly in cases where relatively high intensity laser output is used, such as to ablate or fragment a calculi target of certain size, hardness, or composition. To prevent hazardous consequences such as tissue thermal damage, intracorporeal or surgical-site temperature can be monitored during the procedure to ensure it remains within a safe temperature range. Conventional surgical site temperature control involves real-time monitoring temperature. If the temperature reading reaches or exceeds a safety limit (e.g., a preset threshold), a user (e.g., a physician) can lower the laser output intensity or disable the laser output temporarily. Such manual temperature adjustment have several limitations. First, since the surgical site temperature can rise quickly especially when high laser output is used during the procedure, reducing or shutting off laser output when the temperature reading reaches or exceeds a safety limit may be too late to prevent laser-induced tissue thermal damage. Second, timing of laser output adjustment is critical for preventing tissue damage without compromising ablation or fragmentation efficiency. Manual adjustment of laser output not only puts onus on the operating physician, but may lack precision and predictability, particularly for inexperienced physicians. Third, reducing or shutting off laser output may not produce adequate and fast temperature relief at certain surgical sites or tissue anatomy. In some cases, it is not feasible to shut off or significantly reduce laser output without compromising ablation efficiency. For at least the above reasons, the present inventors have recognized an unmet need for devices and methods for automatic and more effective temperature control to prevent heat built up at surgical site during a procedure such as a laser lithotripsy procedure.

The present invention describes systems, devices, and methods for automatic control of surgical device settings based on tissue whitening detected from images or video frames of at least a portion of the surgical site. An exemplary endoscopic surgical system comprises an endoscopic surgical device controllably coupled to a medical instrument (e.g., a laser system) and configured to deliver energy (e.g., laser energy) to a surgical site during a procedure, an imaging sensor configured to generate images or video frames of at least a portion of the surgical site during the procedure, and a controller circuit configured to analyze the generated images or video frames to determine whether a degree of heat built up in a first target (e.g., tissue) at the surgical site exceeds a predetermined threshold. As there is a positive correlation between the degree of tissue whitening and the degree of heat buildup, the determination of the degree of heat buildup can be based on whether tissue whitening has occurred and the degree of tissue whitening at the first target. Based on such determination, the controller circuit can determine whether to adjust at least one operating parameter associated with the endoscopic surgical system so as to achieve or maintain a treatment effect of a second target (e.g., a calculi target) at the surgical site different from the first target, while avoid damaging the first target during the procedure.

The systems, devices, and methods according to various embodiments discussed herein improve real-time surgical site temperature control during a laser endoscopy procedure. Features described herein may further be used in regard to an endoscope, laser surgery, laser lithotripsy or ultrasound lithotripsy, irradiation parameter settings, and/or spectroscopy. Examples of targets and applications may include laser lithotripsy or ultrasound lithotripsy of renal calculi and laser incision or vaporization of soft tissue. In an example of endoscopic system that incorporate the features as described herein, surgical site conditions such as excessive heat buildup may be detected by analyzing images or video frames of at least a portion of the surgical site taken during the procedure, and identifying from the images or video frames tissue whitening as early signs of tissue thermal damage. Compared to conventional display of temperature measurement, the image-based surgical site temperature control as described in the present document allows tissue whitening to be detected at an early stage of surgical site heat buildup. Identification of tissue whitening allows for early and more effective preventive actions to be taken well before the temperature rises to a critical level, thereby preventing tissue thermal damage and improving patient safety.

The present document describes various temperature control means to regulate surgical site temperature, such as keeping the temperature below a critical level or within a desired safety range. In an example, laser output intensity or one or more laser irradiation parameters (e.g., one or more laser pulse parameters such as, power, duration, frequency, or pulse shape, exposure time, or firing angle) may be adjusted. In some examples, pulse sequencing (which represents a temporal distribution of laser pulses within a specific time) can be adjusted or randomized to produce a non-equilibrium state of irrigation flow. Such a flow can help prevent or reduce the likelihood of vaporized bubbles (induced by the laser pulses) from continuously striking the same area of the tissue which may exacerbate local heat buildup. For example, adjusting the laser setting(s) may result in a non-equilibrium flow that causes bubbles created by the laser energy to strike a wide range of tissue areas rather than a single tissue area at which tissue-whitening is observed. In one embodiment, the non-equilibrium irrigation flow can promote collapses of the vaporized bubbles at or near the surgical site. In addition or alternative to adjusting the laser output setting, regulating the irrigation inflow into the surgical site and/or outflow (suction) out of the surgical site may also put the surgical site temperature under control. In some embodiments, irrigant can be treated (e.g., chilled) before flowing into the surgical site to more quickly and effectively reduce the surgical site temperature. One or more of such temperature control means may be optimized based on the surgical site conditions. For example, based on the tissue pressure at or near the surgical site, irrigation or suction flow may be adjusted to achieve or maintain a desired environment pressure at or near the surgical site while producing the temperature control effect. In some examples, multiple temperature control means (e.g., adjusting laser output or irradiation parameter, adjusting irrigation or suction flow, or providing irrigant treatment) may be combined or arranged to form a tiered temperature control strategy based on the surgical site condition. Compared to conventional approach that focuses on controlling laser output, the various temperature control means and the tiered temperature control strategy as discussed in this document advantageously allows for more versatile control of surgical site temperature in accordance with the surgical site conditions. Using alternative temperature control means such as irrigation or suction flow and irrigant treatment can help avoid discontinuation or substantial reduction of laser energy output during a laser lithotripsy procedure, such that laser therapy efficacy would not be significantly compromised. Consequently, more precise and faster temperature control and improved laser therapy efficacy and tissue safety may be achieved.

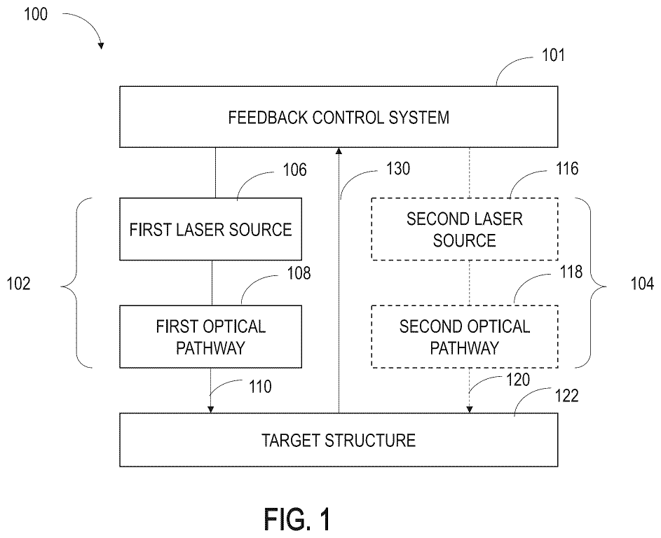

FIG. 1 is a block diagram illustrating an example of a laser energy delivery system 100 configured to provide laser treatment to an anatomical target at or near a surgical site 122 in a body of a subject, such as anatomical structure (e.g., soft tissue, hard tissue, or abnormal such as cancerous tissue) or calculi structure (e.g., kidney or pancreobiliary or gallbladder stone). In some examples, the laser energy delivery system 100 may deliver precisely controlled therapeutic treatment of tissue or other anatomical structures (e.g., tissue ablation, coagulation, vaporization, or the like) or treatment of non-anatomical structures (e.g., ablation or dusting of calculi structures).

The laser energy delivery system 100 can include a feedback control system 101, and at least one laser system 102 in operative communication with the feedback control system 101. By way of example and not limitation, FIG. 1 shows the laser feedback system connected to a first laser system 102 and optionally (shown in dotted lines) to a second laser system 104. Additional laser systems are contemplated within the scope of the present disclosure. The first laser system 102 may include a first laser source 106, and associated components such as power supply, display, cooling systems and the like. The first laser system 102 may also include a first optical pathway 108 operatively coupled with the first laser source 106. In an example, the first optical pathway 108 includes an optical fiber. The first optical pathway 108 may be configured to transmit laser beams from the first laser source 106 to the target structure at or near a surgical site 122.

The feedback control system 101 may receive feedback signals 130 from the target. Various feedback signals may be used to control laser delivery, laser energy output, and/or other system parameters to improve therapy efficacy and to achieve or maintain a desired condition such as a desired temperature at or near the surgical site to prevent or reduce the severity of laser-induced tissue thermal damage. In an example, the feedback signals 130 may include signals indicative of surgical site condition such as a temperature or a pressure at or near the surgical site during the procedure. In an example, the feedback signals 130 may include an acoustic signal produced by a laser pulse propagating through the media (e.g., liquid and vapor), projecting to the target and causing the target to vibrate. In another example, the feedback signals 130 may include reflected electromagnetic signal (e.g., reflected illumination light emitted from a light source). In yet another example, the feedback signals 130 may include reflected laser signal. The feedback control system 101 may analyze the feedback signals 130, generate signal properties from the feedback signals 130, and control laser output (e.g., energy intensity, or other laser irradiation parameters such as power, duration, frequency, or pulse shape, exposure time, or firing angle) or other system parameters according to the signal properties. In an example, the feedback signals 130 may include images or video frames of at least a portion of the surgical site such as generated by an imaging sensor during a procedure. The feedback control system 101 may analyze the images or the video frames to determine whether a degree of heat built up in a first target (e.g., tissue) at the surgical site exceeds a predetermined threshold. The predetermined threshold, which can be different for different types of tissue, can be represented by a threshold temperature (e.g., 42° C.) that starts to cause undesirable clinical effects on the tissue. As there is a positive correlation between the degree of tissue whitening and the degree of heat buildup, the degree of heat buildup can be inferred by evaluating the degree tissue whitening in the first target, and the predetermined threshold for heat buildup can be represented by a threshold degree of tissue whitening. Based on the determination of the degree of heat buildup (e.g., tissue whitening at the first target), the feedback control system 101 may adjust laser output or laser delivery and/or other system parameters to achieve or maintain a treatment effect of a second target (e.g., a calculi target) at the surgical site different from the first target, while avoid damaging the first target during the procedure. In an example, the first target can be tissue in a urinary system, and the second target can be a renal calculi target. The feedback control system 101 may adjust laser output or laser delivery and/or other system parameters to achieve or maintain a desired surgical site condition, such as a desired surgical site temperature during a lithotripsy procedure to prevent or reduce the severity of laser-induced tissue thermal damage, while maintain a treatment effect of ablating or fragmenting the renal calculi target.

As shown in FIG. 1, based on the analysis of the feedback signals 130, the feedback control system 101 may control the first laser system 102 and/or the second laser system 104 to generate suitable laser outputs to achieve a desired therapeutic effect and to achieve or maintain a desired condition such as a desired temperature at or near the surgical site to prevent or reduce the severity of laser-induced tissue thermal damage. For instance, the feedback control system 101 may monitor properties of the target structure during a therapeutic procedure (e.g., ablating calculi such as kidney stones into smaller fragments) to determine if the tissue was suitably ablated prior to another therapeutic procedure (e.g., coagulation of blood vessels).

In an example, the first laser source 106 may be configured to provide a first output 110. The first output 110 may extend over a first wavelength range, such as one that corresponds to a portion of the absorption spectrum of the target structure. The first output 110 may provide effective ablation and/or carbonation of the target structure since the first output 110 is over a wavelength range that corresponds to the absorption spectrum of the tissue.

In an example, the first laser source 106 may be configured such that the first output 110 emitted at the first wavelength range corresponds to high absorption (e.g., exceeding about 250 cm⁻¹) of the incident first output 110 by the tissue. In example aspects, the first laser source 106 may emit first output 110 between about 1900 nanometers (nm) and about 3000 nm (e.g., corresponding to high absorption by water) and/or between about 400 nm and about 520 nm (e.g., corresponding to high absorption by oxy-hemoglobin and/or deoxy-hemoglobin). Appreciably, there are two main mechanisms of light interaction with a tissue: absorption and scattering. When the absorption of a tissue is high (absorption coefficient exceeding 250 cm⁻¹) the first absorption mechanism dominates, and when the absorption is low (absorption coefficient less than 250 cm⁻¹), for example lasers at 800-1100 nm wavelength range, the scattering mechanism dominates.

Various commercially available medical-grade laser systems may be suitable for the first laser source 106. For instance, semiconductor lasers such as InXGa1-XN semiconductor lasers providing the first output 110 in the first wavelength range of about 515 nm and about 520 nm or between about 370 nm and about 493 nm may be used. Alternatively, infrared (IR) lasers such as those summarized in Table 1 below may be used.

TABLE 1
Example List of suitable IR lasers

| | | | Optical |
|---|---|---|---|
| | | Absorption | Penetration |
| | Wavelength | Coefficient | Depth |
| Laser | λ (mm) | μ₂ (cm⁻¹) | δ (μm) |
| Thulium fiber laser: | 1908 | 88/150 | 114/67 |
| Thulium fiber laser: | 1940 | 120/135 | 83/75 |
| Thulium:YAG: | 2010 | 62/60 | 161/167 |
| Holmium:YAG: | 2120 | 24/24 | 417/417 |
| Erbium:YAG: | 2940 | 12,000/1,000 | 1/10 |

The optional second laser system 104 may include a second laser source 116 for providing a second output 120, and associated components, such as power supply, display, cooling systems and the like. The second laser system 104 may either be operatively separated from or, in the alternative, operatively coupled to the first laser source 106. In some embodiments, the second laser system 104 may include a second optical pathway 118 (separate from the first optical pathway 108) operatively coupled to the second laser source 116 for transmitting the second output 120. Alternatively, the first optical pathway 108 may be configured to transmit both the first output 110 and the second output 120.

In certain aspects, the second output 120 may extend over a second wavelength range, distinct from the first wavelength range. Accordingly, there may not be any overlap between the first wavelength range and the second wavelength range. Alternatively, the first wavelength range and the second wavelength range may have at least a partial overlap with each other. In advantageous aspects of the present disclosure, the second wavelength range may not correspond to portions of the absorption spectrum of the target structure where incident radiation is strongly absorbed by tissue that has not been previously ablated or carbonized. In some such aspects, the second output 120 may advantageously not ablate uncarbonized tissue. In another embodiment, the second output 120 may ablate carbonized tissue that has been previously ablated. In additional embodiments, the second output 120 may provide additional therapeutic effects. For instance, the second output 120 may be more suitable for coagulating tissue or blood vessels.

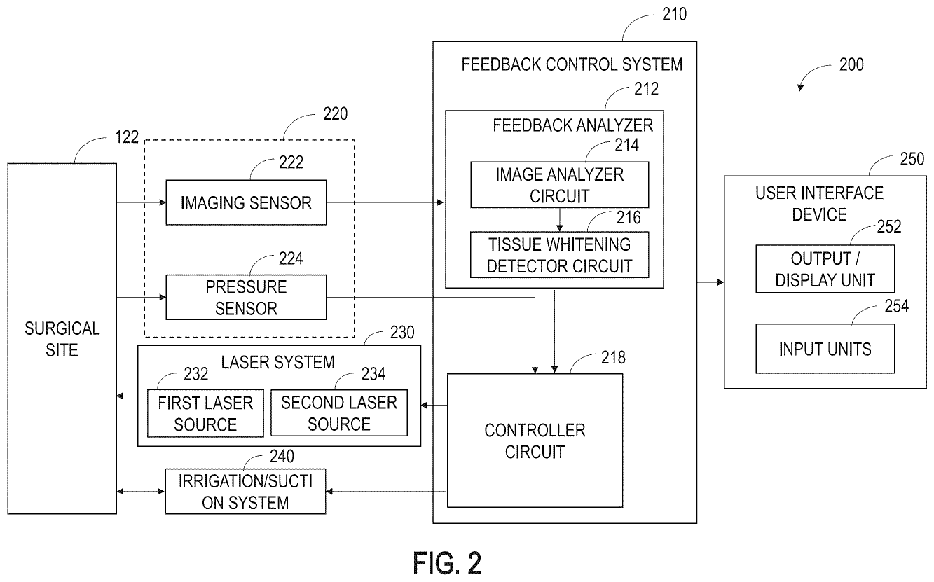

FIG. 2 is a block diagram illustrating an endoscopic surgical system 200 with automatic surgical site condition control, and at least a portion of the environment in which the system 200 may operate. The system 200 can be an embodiment of the laser energy delivery system 100, or a lithotripsy system that may be used for destructing hardened masses like kidney stones, bezoars, gallstone, among other calculi structures. The system 200 may monitor and control conditions at or around the surgical site 122 during a laser procedure, such as to keep the surgical site temperature at substantially a desired level during a procedure to prevent or reduce the severity of laser-induced tissue thermal damage. In this document, the term “substantially” means ±10%, and in some embodiments, ±5%.

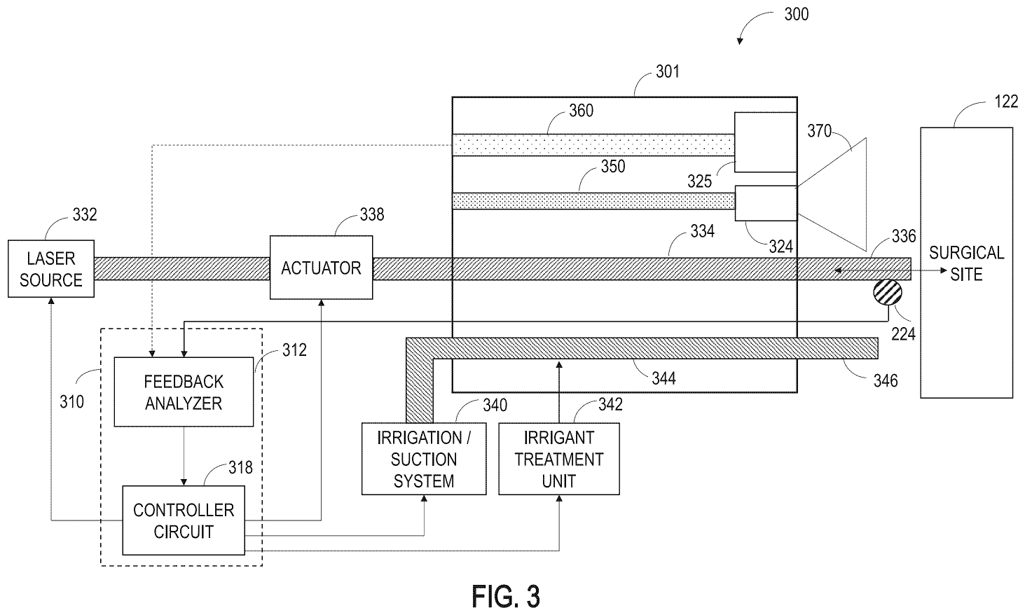

The endoscopic surgical system 200 can includes a feedback control system 210, one or more sensors 220, a laser system 230, an irrigation and/or suction system 240, and a user interface device 250. The feedback control system 210, which is an embodiment of the feedback control system 101 of the laser energy delivery system 100, can include a feedback analyzer 212 and a controller circuit 218. According to example embodiments, the feedback control system 210 may include processors, such as microprocessors, digital signal processors (DSPs), application specific integrated circuits (ASICs), field programmable gate arrays (FPGAs), or any other equivalent integrated or discrete logic circuitry, as well as any combinations of such components for performing one or more of the functions attributed to the feedback control system 210. The feedback analyzer 212 may be communicatively coupled to one or more sensors 220, receive therefrom feedback signals, and analyze the feedback signals to generate one or more signal properties that may be used for controlling surgical site conditions. In an example as illustrated in FIG. 2, the one or more sensors 220 can include an imaging sensor 222 configured to generate images or video frames of at least a portion of the surgical site 122 during the procedure. The imaging sensor 222 can be included in an imaging system that further includes a lens system. Examples of the image sensor 222 can include a CCD or CMOS camera sensitive in ultraviolet (UV), visible (VIS) or infrared (IR) wavelengths. The imaging sensor 222 can be located a distal portion of an endoscope for use during the procedure, an example of which is illustrated in FIG. 3. The imaging sensor 222 can generate images or video frames at different times. The one or more sensors 220 can additionally include a pressure sensor 224 to sense surgical site pressure during the procedure.

The images or video frames of the surgical site can be transmitted to the feedback analyzer 212. The feedback analyzer 212 can include an image analyzer circuit 214 and a tissue whitening detector circuit 216. The image analyzer circuit 214 can process the images or video frames to detect image features therefrom indicative of a change in tissue color over time. The tissue whitening detector circuit 216 can detect an indication of tissue whitening using the detected image features. The tissue whitening can be indicative of a degree of heat built up in a first target (e.g., tissue) at the surgical site. In an example, the image analyzer circuit 214 can compare images or video frames taken at different times during the procedure, and identify a change in intensity of one or more characteristic color components (e.g., CMOS or CCD color components) at a registered location in the images or video frames. The registered location, which corresponds to a specific tissue site, can be identified by the user. In an example, an aiming beam can be used to assist in identifying the registered location corresponding to a specific tissue site, as to be discussed below. The tissue whitening detector circuit 216 can compare the intensity of one or more characteristic color components (e.g., RGB values) of the registered location to respective threshold values (e.g., threshold RGB values, RGB_{TH}) for a “white” color (RGB (255, 255, 255)) or a pre-determined “whitened” color (e.g., RGB (240, 240, 240)). If the intensity values (e.g., from 0 to 255) of one or more color components are close enough to the “white” color within a specified margin (e.g., RGB values within a range of (240-255, 240-255, 240-255), then a tissue whitening is deemed detected. In some examples, the image analyzer circuit 214 can compare consecutively captured images, or perform a frame-to-frame comparison of the video frames, and determine a color intensity change rate towards the “white” color or the pre-determined “whitened” color at the registered location. The tissue whitening detector circuit 216 can compare the color intensity change rate to a rate threshold, and determine the presence of tissue whitening if the color intensity change rate exceeds the rate threshold. The tissue whitening thus detected indicates that the degree of heat built up in the tissue at the surgical site exceeds a predetermined threshold corresponding to RGB_{TH} or the threshold color intensity change rate.

The controller circuit 218 may be coupled by wired or wireless connections to the feedback analyzer 212. The controller circuit 218 can generate a control signal to adjust an operating parameter associated with the system 200 to achieve or maintain substantially a desired temperature (e.g., ±10%, or in some embodiments, ±5%) at the surgical site in response to the identified tissue whitening. In an example, the tissue whitening detector circuit 216 may determine a degree of tissue whitening ΔRGB based on a difference between the intensity values of the color components (e.g., RGB values) of an image or video frame taken at time t (RGBₜ) and the threshold values RGB_{TH} for the “white” color (RGB (255, 255, 255)) or the pre-determined “whitened” color (e.g., RGB (240, 240, 240)), that is, ΔRGB=RGBₜ−RGB_{TH}. A smaller ΔRGB value indicates a higher degree of tissue whitening (i.e., closer to “white” or pre determined “whitened” color), thus a higher temperature at the surgical site and a higher risk of tissue thermal damage. In another example, the tissue whitening detector circuit 216 may determine a rate of tissue whitening ΔRGB/Δt (i.e., the amount of color intensity change towards the “white” color or the pre-determined “whitened” color in a unit time). A higher tissue whitening rate ΔRGB/Δt indicates a faster heat buildup at the surgical site thus a higher risk of tissue thermal damage. The controller circuit 218 can determine an aggressiveness of parameter adjustment (e.g., by adjusting the laser output setting, or irrigation and/or suction flow rate, or irrigant temperature control, etc.) based on the degree of tissue whitening ΔRGB, or the rate of tissue whitening ΔRGB/Δt. For example, if a higher degree of tissue whitening or a higher rate tissue whitening rate (i.e., a faster tissue whitening) is detected, the controller circuit 218 can provide more aggressive adjustments of one or more system parameters (e.g., more significant reduction in laser output, a higher irrigation flow rate and/or a higher suction flow rate, or more chilling of the irrigant before flowing into the surgical site) to bring the surgical site temperature under control.

The laser system 230, which is an example of the laser system 102 or the laser system 104 as shown in FIG. 1, can include a laser source (such as the first laser source 106) and an optical pathway (such as the first optical pathway 108) for directing the laser energy to the surgical site. The laser source can generate laser energy in accordance with a laser output intensity or one or more laser irradiation parameters (e.g., one or more laser pulse parameters such as, power, duration, frequency, or pulse shape, exposure time, or firing angle). At least some of such laser parameters are programmable or adjustable either automatically such as by the controller circuit 218, or manually by a user via the user interface device 250. In response to the indication of the tissue whitening (as detected by the tissue whitening detector circuit 216), the controller circuit 218 can automatically adjust a laser output setting in accordance with one or more of the degree of tissue whitening ΔRGB, or the rate of tissue whitening ΔRGB/Δt. For example, the controller circuit 218 can automatically reduce average power of laser pulses delivered to the surgical site, such as by reducing one or more of a pulse width of a laser pulse, a peak power of a laser pulse, or a pulse frequency representing a number of laser pulses per unit time. Reducing the average power of laser pulses can decrease the laser-induced heating effect at or near the surgical site, thereby preventing tissue thermal damage and improving patient safety during the procedure.

In addition or alternative to adjusting one or more laser output parameters, the controller circuit 218 can automatically select one of a plurality of pre-determined laser output settings or pulse profiles with different energy output levels in accordance with the degree of tissue whitening ΔRGB, or the tissue whitening rate ΔRGB/Δt. In an example, the controller circuit 218 can automatically toggle between a first “high output” setting and a second “low output” setting with respective pre-determined parameter values. The “low output” setting has a lower average power than the “high output” setting. When the degree of tissue whitening ΔRGB or the rate of tissue whitening ΔRGB/Δt exceed respective thresholds, the “low output” setting will be automatically selected.

In some examples, the laser system 230 can include a first laser source 232 and a different second laser source 234. The first laser source 232 may generate treatment laser energy directed to the target at the surgical site through an optical pathway, such as the first optical pathway 108. Examples of the first laser source 232 can include a thulium laser, Ho:YAG, Nd:YAG, and CO₂, among others. The second laser source 234 may be optically coupled to the same or a different optical pathway, such as the second optical pathway 118. The second laser source 234 may generate an aiming beam directed to the target through the same or a different optical pathways. For example, the wavelength of the aiming beam may be in the range of 500-550 nm. In some examples, the second laser source 234 may emit at least two different aiming beams with different characteristics, such as one or more of a wavelength, a power level, or an emitting pattern. For example, the first aiming beam can have a wavelength in the range of 500 nm to 550 nm while the second aiming beam can have a wavelength in the range of 635 nm to 690 nm. The characteristics of the different aiming beams may be selected based on the visibility of the aiming beams in the images or video frames of the surgical site.

The aiming beam may be emitted when the target is illuminated by the illuminated light (such as the illumination light source 324 as shown in FIG. 3). In some examples, the aiming beam may be generated using a light source different than the second laser source 234. The light source can generate an aiming beam with a distinct color to distinguish from the illumined background of the surgical site. By way of example and not limitation, the aiming beam can be a green light in the range of approximately 520 nm, or can be a red light in the range of approximately 620 nm in another example.

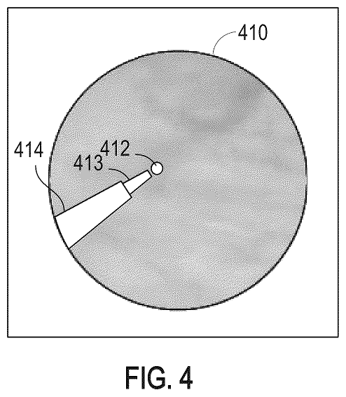

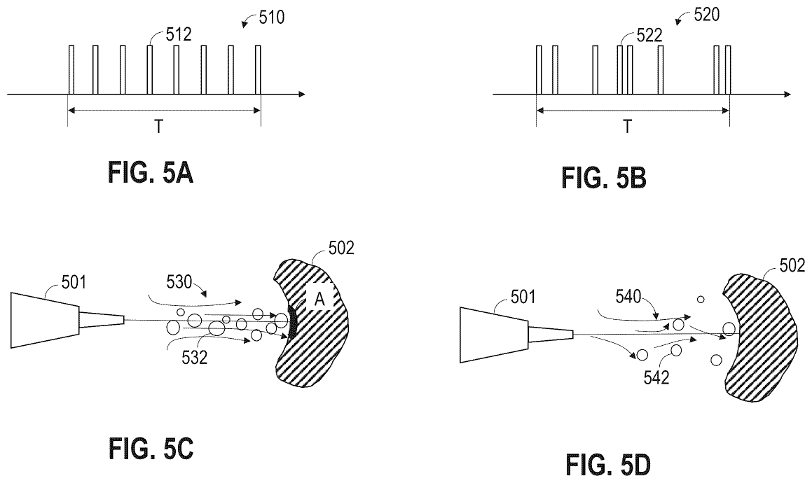

The aiming beam incident on the tissue at the surgical site can be captured by the imaging sensor 222, and shown as an aiming beam footprint in the images or video frames. FIG. 4 illustrates an endoscopic image 410 including a graphical representation of the illuminated target site that falls within the field of view of the imaging sensor 222, and a circular-shaped aiming beam footprint 412. Also shown in the image 410 are an image of the distal tip 413 of an optical pathway (e.g., the distal end 336 of the optical pathway 334 as shown in FIG. 3) and an image of a distal portion 414 of the endoscope (such as the endoscope 301 as shown in FIG. 3). When a colored (e.g., green) aiming beam is used, the aiming beam footprint 412 can also appear green in the endoscopic image 410. The tissue whitening detector circuit 216 can identify the aiming beam footprint 412 from each of a sequence of images or video frames of the surgical site, and detect tissue whitening from a portion of the images or video frames at or in a vicinity of the aiming beam footprint. In one embodiment, when the tissue becomes “whitened”, a colored (e.g., green or red) aiming beam incident on the “whitened” tissue can appear brighter in the image or video frames than on the “un-whitened” tissue. For example, for a green aiming beam, a brighter green footprint may indicate a higher degree of tissue whitening than a dull green footprint. The brightness of the color of the footprint can be measured as color intensities or saturation (e.g., RGB values). In an example, the tissue whitening detector circuit 216 can detect tissue whitening (and further determine the degree of tissue whitening ΔRGB and/or the tissue whitening rate ΔRGB/Δt) based on an increase in color intensity at or in a vicinity of the aiming beam footprint. Based on such aiming-beam indicated tissue whitening, the controller circuit 218 can adjust one or more system parameters to bring the surgical site temperature under control.

As stated above, in response to the detection of tissue whitening (by the tissue whitening detector circuit 216), the controller circuit 218 can automatically adjust a laser output setting including one or more laser irradiation parameters in accordance with the degree of tissue whitening ΔRGB or the rate of tissue whitening ΔRGB/Δt to lower the surgical site temperature. Maintaining a desired surgical site condition (e.g., temperature) can help achieve or maintain a desired treatment effect of a treatment target at the surgical site (e.g., ablation or fragmentation of a calculi target), while avoid damaging the tissue in the vicinity of the treatment target during the procedure. One example of the laser irradiation parameters to be adjusted for controlling surgical site temperature is a pulse sequencing (also referred to as pulse profile). Pulse sequencing represents timings, or temporal distributions, of laser pulses within a specific time. Adjusting the pulse sequencing can alter heat distribution within the surgical space such by altering the formation, flow, and quantity of vaporized bubbles produced by the pulsed laser beam. Such vaporized bubbles can be formed when the laser pulses propagate through the surgical space into contact with tissue walls. Tissue areas in contact with the vaporized bubbles tend to be overheated by the bubbles. For example, in some cases, a temperature rise of 25° C.-30° C. may occur at tissue areas which bubbles contact. Additionally, temperature rise of tissue at or near the surgical site may also be affected by the bubble size and the position of optical pathway (e.g., the fiber-tissue distance). Accordingly, reducing the size and number of vaporized bubbles and/or redistributing the bubbles across a wide range of tissue areas (to prevent bubbles from constantly contacting with the same tissue area) can help lower the surgical site temperature and prevent or reduce the severity of tissue thermal damage.

A constant pulse sequencing of the laser may facilitate the bubbles travelling in an equilibrium state to a constant point of contact with the tissue wall. Under such equilibrium state, a predictable turbulent flow of bubbles toward the same tissue area can constantly strike the tissue area and cause heat buildup. To mitigate the delirious effects of a single point of tissue continually absorbing laser induced heat via the bubble stream, the controller circuit 218 responds to identification of tissue whitening within the images or video frames by generating a control signal to the first laser source 232 to adjust the laser output settings, including the laser pulse sequencing, to prevent bubbles from traveling in an equilibrium state and continuously striking the same area of tissue. In particular, altering the pulse sequencing (i.e., changing the temporal distribution of laser pulses within a specified time) can produce a non-equilibrium, or more chaotic and unpredictable irrigation flow in which the bubbles strike a wide range of tissue areas rather than the single tissue area at which tissue whitening would be observed. The pulse sequencing may be altered periodically or at user specified times. FIGS. 5A-5D illustrate an example of changing the laser pulse sequencing from an first pulse profile 510 (in FIG. 5A) to a different second pulse profile 520 (in FIG. 5B) within a given time period T, and the resultant irrigation flow and bubble state. The second pulse profile 520 is characterized by a different, randomized distribution of laser pulses 522 than the distribution of pulses 512 of the first pulse profile 510. In an example, a random number generator can be used to determine the timings for the laser pulses 522 within the given time period T. FIG. 5C illustrates an equilibrium state produced by laser pulses, in accordance with the first pulse profile 510, emitted from a distal end 501 into the surgical space of tissue 502. The equilibrium state is characterized by a predictable, steady turbulent flow 530, in which vaporized bubbles 532 flow toward site “A” of the tissue 502, and constantly strike the same tissue site “A”, causing heat buildup therein. In contrast, FIG. 5D illustrates a non-equilibrium state produced by laser pulses delivered in accordance with the second pulse profile 520. The non-equilibrium state is characterized by a more unpredictable irrigation flow 540 with fewer vaporized bubbles 542 due to a higher chance of bubble collapse. The bubbles 542 in the non-equilibrium state may strike a wide range of areas of the tissue 502 rather than the single tissue area “A”, thereby preventing or reducing the severity of tissue thermal damage at the surgical site.

In some examples, in response to the indication of the tissue whitening (as detected by the tissue whitening detector circuit 216), the controller circuit 218 can generate a control signal to an actuator coupled to an optical pathway (e.g., a laser fiber) of the laser system 230 to adjust the position or orientation of an adjustable distal portion (laser firing portion) of the optical pathway relative to the anatomical target at or near the surgical site. The adjustment of the position or orientation of the distal portion of the optical pathway can include adjusting a distance between the distal portion and the anatomical target (the “fiber-target” distance), or an aiming angle of the distal portion with respect to the anatomical target, in accordance with one or more of the degree of tissue whitening ΔRGB, or the rate of tissue whitening ΔRGB/Δt provided by the tissue whitening detector circuit 216. For example, the controller circuit 218 can automatically, via the actuator, move the distal portion of the optical pathway farther away from the surgical site (i.e., increase the fiber-target distance) and/or rotating the distal portion of the optical pathway to aim the laser away from the surgical site (to increase the aiming angle). By increasing the fiber-target distance and/or increasing the aiming angle, the density of the laser energy incident on the surgical site and the laser-induced heat transferred into the surgical site can be reduced.

In certain procedures (e.g., lithotripsy) where the intended target to be treated is not anatomical tissue or organs but masses such as calculi structures at the at the surgical site, an identification of tissue whitening can be an indication of poor targeting of the laser fiber. Adjusting the position or orientation of the distal portion of the optical pathway (to change the aiming angle, or the “fiber-target” distance) such as via the actuator responsive to tissue whitening can redirect the laser energy to the intended calculi target to improve therapy efficacy while preventing tissue thermal damage.

The irrigation and/or suction system 240 can include one or more irrigation and/or suction sources that can provide a flow of irrigation fluid (also referred to as irrigant, e.g., saline solution) to the surgical site through at least one irrigation channel such as included in an endoscope during the procedure. The irrigation fluid can facilitate removal of the tissue debris, stone fragments, and other unwanted matters through a suction channel. The irrigation flow also has a cooling effect on the tissue at or near the surgical site and the surgical tools (e.g., endoscopic tissue removal device), and can help dissipate the heat generated during ablation of calculi. Examples of the irrigation and/or suction system 240 are discussed below with reference to FIG. 3.

In some examples, in response to the indication of the tissue whitening (as detected by the tissue whitening detector circuit 216), the controller circuit 218 can automatically adjust one or more irrigation parameters, such as an irrigation flow or a suction flow, in accordance with the degree of tissue whitening ΔRGB, or the tissue whitening rate ΔRGB/Δt provided by the tissue whitening detector circuit 216. For example, the controller circuit 218 can automatically increase the irrigation flow from the irrigation source to the surgical site to increase convective heat transfer. Additionally or alternatively, the controller circuit 218 can automatically increase the suction flow (or suction pressure) to more effectively withdraw the fluid away from the surgical site to improve heat dissipation and reduce the surgical site temperature.

The application of irrigation or suction flow for controlling surgical site temperature can fluctuate the pressure at or near the surgical site. For example, irrigation flow into the surgical site would generally increase the surgical site pressure (positive pressure change), while a suction pressure (i.e., outflow) would generally decrease the surgical site pressure (negative pressure change). Such irrigation and/or suction-induced positive or negative pressure changes, if not property regulated, may be harmful to tissue or organs at or near the surgical site. To keep the pressure of the anatomical environment under control during the procedure and to avoid or reduce pressure-related tissue damage, the system 200 can include a pressure sensor 224 to sense surgical site pressure during the procedure. When the degree of tissue whitening ΔRGB, or the tissue whitening rate ΔRGB/Δt, satisfy respective conditions (e.g., ΔRGB is shorter than a threshold, or ΔRGB/Δt exceeds a threshold), the controller circuit 218 can selectively activate or adjust irrigation flow or the suction flow based on the measured surgical site pressure (P). For example, as an increase in irrigation flow into the surgical site may introduce a positive pressure change at or near the surgical site, if the measured surgical site pressure exceeds a predetermine or user-specified upper pressure limit (also referred to as a maximal allowable pressure) Pₘₐₓ(P>Pₘₐₓ), then the controller circuit 218 can increase the suction flow to reduce the surgical site temperature but avoid increasing the irrigation flow to prevent further increase in surgical site pressure. For example, the irrigation flow can be maintained at its current rate or set to a reduced rate, or temporarily deactivated. The increased suction flow may also help reduce the surgical site pressure to a level within the desired pressure range. If the measured surgical site pressure is within a desired pressure range between the upper pressure limit Pₘₐₓ and a lower pressure limit Pₘᵢₙ(Pₘᵢₙ<P<Pₘₐₓ), then the controller circuit 218 can increase one or both of the irrigation flow and the suction flow to reduce the surgical site temperature. As an increase in suction flow may introduce a negative pressure change at or near the surgical site, if the measured surgical site pressure falls below the lower pressure limit Pₘᵢₙ(P<Pₘᵢₙ), then the controller circuit 218 can increase the irrigation flow into the surgical site to reduce the surgical site temperature, but avoid increasing the suction flow to prevent further decrease in surgical site pressure. For example, the suction flow can be maintained at its current rate or set to a reduced rate, or temporarily deactivated. The increased irrigation flow may also help increase the surgical site pressure to a level within the desired pressure range.

In some examples, the irrigation and/or suction system 240 can include an irrigant treatment unit that can adjust the temperature of the irrigation fluid (irrigant) before being applied to the surgical site. In some examples, in response to the indication of the tissue whitening (as detected by the tissue whitening detector circuit 216), the controller circuit 218 can generate a control signal to the irrigant treatment unit to alter the temperature of the irrigant in accordance with one or more of the degree of tissue whitening ΔRGB, or the rate of tissue whitening ΔRGB/Δt provided by the tissue whitening detector circuit 216. In an example, the irrigant treatment unit can include a cooling system (e.g., a radiator, or an in-line chiller) that can, under the control of the controller circuit 218, cool the irrigant before reaching the surgical site. In another example, the irrigant treatment unit includes a fluid mixer that can, under the control of the controller circuit 218, mix at least two irrigant of different temperatures before reaching the surgical site. The cooled irrigate via the cooling system or the mixed irrigant via the fluid mixer, when applied to the surgical site can improve convective heat transfer therein and effectively and efficiently reduce the surgical site temperature.

In some examples, the controller circuit 218 can maintain the surgical site temperature at substantially a desired level or range in accordance with a temperature management plan. The temperature management plan can include a prioritized order of two or more temperature control means described above, including, for example, changing a laser output setting or one or more laser irradiation parameters, adjusting the position or orientation of the distal portion of the optical pathway (e.g., a laser fiber), activating or adjusting an irrigation flow into the surgical site and/or a suction flow away from the surgical site, or altering the temperature of the irrigant before being applied to the surgical site, among other means. The temperature management plan can be programmed or modified by a user, such as via the user interface device 250. The order of the temperature control means can be determined based on the availability (e.g., irrigant cooling system), efficiency of temperature control, or potential adverse effects on the surgical site. In an example, the temperature management plan may be programmed with a bias toward maintaining an optimal or a user-selected laser output setting, while adjusting other device settings (e.g., position or orientation of the distal portion of the optical pathway, irrigation and/or suction flows, irrigant temperature) suitable for managing surgical site temperature. Maintaining the laser output setting can be desirable during a laser lithotripsy procedure to reduce procedure time and ensure therapy efficacy and efficiency. Additionally, adjusting laser output setting (e.g., reducing average laser power) may have a slow effect on the surgical site temperature. In another example, to prevent irrigation and/or suction-induced pressure fluctuation at or near the surgical site, the temperature management plan may be programmed such that irrigant temperature control (e.g., chill the irrigant before being applied to the surgical site) can be used before an attempt to adjust the irrigation or suction flow. For example, in response to the indication of the tissue whitening (as detected by the tissue whitening detector circuit 216), the controller circuit 218 can first generate a control signal to the irrigant treatment unit of the irrigation and/or suction system 240 to chill the irrigant before being applied to the surgical site. The feedback control system 210 may then re-evaluate images or video frames to determine whether tissue whitening still persists or worsens; and if so, the controller circuit 218 may generate a control signal to the irrigation and/or suction system 240 to increase the irrigation flow and/or the suction flow to reduce the surgical site temperature. The choice between, or the order of applying, irrigation flow and suction flow can be based on the surgical site pressure, as discussed above. For example, to keep the surgical site temperature at substantially a desired level or range during the procedure, the system may compare the current surgical site pressure, P, to the pre-determine or user-specified upper pressure limit Pₘₐₓ. If the current surgical site pressure P is substantially below Pₘₐₓ (e.g., the difference between P and Pₘₐₓ exceeds a threshold), then an irrigation inflow rate may be increased to increase convective heat transfer via irrigation. Additionally or alternatively, suction flow can be increased to more efficiently take the heat away from the surgical site. In contrast, if the current surgical site pressure P is substantially close to Pₘₐₓ (e.g., within a user-specified or pre-determined margin, e.g., ±10%), then rather than increasing the irrigation inflow rate, the laser output setting may be lowered. In embodiments where suction flow is actively controlled (e.g., via a pump), if the current surgical site pressure P is substantially close to Pₘₐₓ, then the suction flow rate can be increased to decrease the surgical site pressure in lieu of, or in addition to, lowering the laser output setting. While the body tissue can generally regulate some positive pressure changes, many organs are relatively defenseless to negative pressure changes. Accordingly, in some examples, increasing the irrigation flow may be attempted prior to activating or increasing the suction flow.

The feedback control system 210 may then re-evaluate images or video frames to determine whether tissue whitening still persists or worsens; and if so, the controller circuit 218 may generate a control signal to the laser system 230 to adjust the position or orientation of the distal portion of the optical pathway, or to change a laser output setting or one or more laser irradiation parameters. The laser irradiation parameters may be adjusted in a predetermined order or prioritization. For example, an adjustment of pulse shape or an adjustment of pulse sequencing (temporal distribution of laser pulses within a specific time) may be prioritized over an adjustment of average power of laser pulses. The tiered, sequential activation or adjustment of different temperature control means can help maintain a desired surgical site condition (e.g., temperature, pressure) during the procedure without comprising therapy efficacy and efficiency or imposing additional risk of tissue damage at or near the surgical site.

The user interface device 250 may be operatively in communication with the feedback control system. The user interface device 250 can include an output/display unit 254 to display information including, for example, surgical site conditions such as images, pressure, or other information sensed by the sensors 220, feedback signal generated by the feedback analyzer 212 including the detected indication of tissue whitening, the tissue whitening degree ΔRGB, or the tissue whitening rate ΔRGB/Δt, or current device settings such as the laser output setting or irrigation or suction flow rates, etc. The output/display unit 252 can display UI elements including visual elements, alerts, tactile feedback, or any combination thereof. The output/display unit 252 can generate an alert about potentially hazardous condition at or near the surgical site, such as an elevated temperature indicated by tissue whitening, or an elevated surgical site pressure. The alert can be presented in an audible, visible, tactile, or otherwise human-perceptible format.

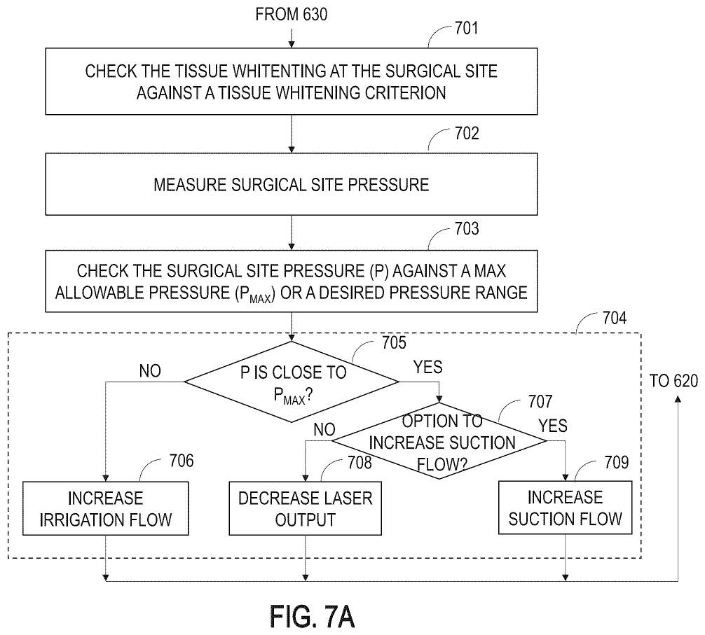

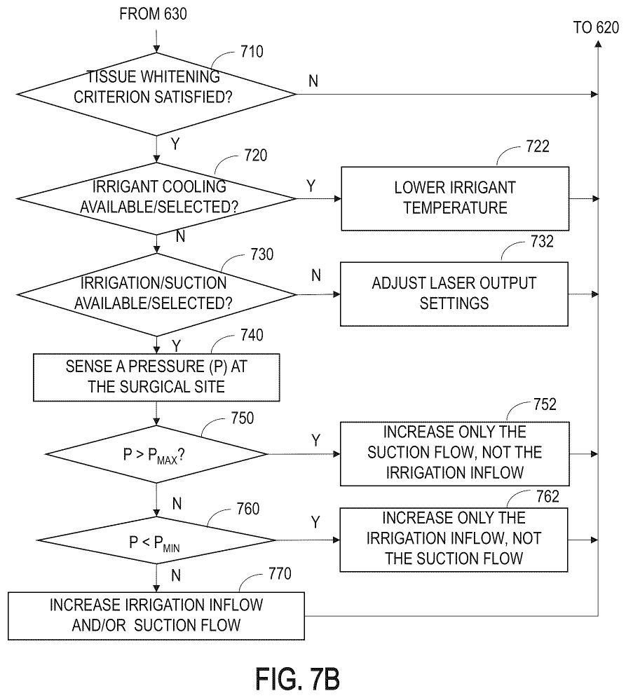

The user interface device 250 can include one or more input units 254 to receive user programming of the device, such as parameter values used for detecting tissue whitening (including, for example, RGB values pro-determined “whitened” color, threshold values for tissue whitening degree ΔRGB and/or tissue whitening rate ΔRGB/Δt). The user input may include adjustment of laser output setting, irrigation or suction flow parameters, among other device parameters for controlling surgical site temperature. In some examples, a user may provide, via one or more input units 254, the temperature management plan that defines a prioritized order of two or more temperature control means as described above. For example, a user may guide the controller circuit 218 to first decrease irrigant temperature (if available) without adjusting laser output of irrigation flow rate. Then, if tissue whitening at the surgical site persists or worsens, then the flow rate can be increased, and/or the irrigant temperature can be decreased. Examples of prioritized means for controlling surgical site temperature are discussed below with reference to FIGS. 7A-7B.

In some examples, the output/display unit 252 may generate a recommendation for taking preventive actions to prevent tissue damage, such as recommended adjustment of laser output or other system parameters. A user may provide an input via the one or more input units 254 to confirm, reject, or modify the recommended adjustment.

FIG. 3 illustrates an example of an endoscopic laser lithotripsy system 300 with automatic surgical site condition control, which can be an example of the endoscopic surgical system 200. The endoscopic laser lithotripsy system 300 may include an endoscope 301, a feedback control system 310, an actuator 338, an irrigation and/or suction system 340, an irrigant treatment unit 342. The endoscope 301 has a proximal portion and an elongate distal portion configured to be inserted into a surgical site of a patient during an endoscopic laser lithotripsy procedure. The endoscope 301 may provide visual inspection or treatment of soft (e.g., non-calcified) or hard (e.g., calcified) tissue as well as for visualizing or breaking up or otherwise treating kidney stones or other stones or other targets. As illustrated in FIG. 3, the endoscope 301 may include or provide visualization and illumination optics, such as a visualization optical pathway 360 and an illumination optical pathway 350, each of which may extend longitudinally along the elongate body of the endoscope 301. An eyepiece or camera or imaging display may be provided at or coupled to the visualization optical pathway 360 to permit user or machine visualization of a target region at or near a distal end of the endoscope 301. The target region may be illuminated by light 370, such as provided by an illumination light source 324 at a proximal end of the illumination optical pathway 350 and emitted from a distal end of the illumination optical pathway 350. The light source 324 can include, for example, a Xenon lamp, a light-emitting diode (LED), a laser diode (LD), or any combination thereof. In an example, the light source 324 may include two or more light sources that emit light having different illumination characteristics, referred to as illumination modes. In an example, the illumination modes may include a white light illumination mode, or a special light illumination mode such as a narrow band imaging mode, an auto fluorescence imaging mode or an infrared imaging mode. A special light illumination can concentrate and intensify specific wavelengths of light, for example, resulting in a better visualization of tissue or other structures at the surgical site.

The lithotripsy system 300 may include or be coupled to at least one laser source 332, which may be an example of the first laser source 106, the second laser source 116, or the laser source included in the laser system 230. The laser source 332 may be mechanically and optically connected to an optical pathway 334, which may include a single optical fiber or a bundle of optical fibers. The optical pathway 334, which is an embodiment of the first optical pathway 108 or the second optical pathway 118, or the optical pathway included in the laser system 230, may be introduced via a proximal access port to extend within a working channel or other longitudinal passage or lumen of the endoscope 301 or similar instrument.

In some examples, the laser source 332 can include a first laser source to generate a treatment beam (such as the first laser source 232) and a second different laser source to generate an aiming beam (such as the second laser source 234). The treatment beam and the aiming beam can be directed to the target through the same or a different optical pathways. In some examples, the aiming beam may be generated using a light source different than the second laser source. As described above with reference to FIG. 2, the aiming beam may have a distinct color (e.g., green or red) to distinguish from the illumined background of the surgical site.

The lithotripsy system 300 can include a camera or imaging device 325. The camera or imaging device 325 can include an imaging sensor (such as the imaging sensor 222) that can generate an imaging signal of the target in response to electromagnetic radiation (e.g., illumination light 370) of the target at or near the surgical site. The imaging signal may be transmitted through the optical pathway 360, or alternatively through the optical pathway 334, to the feedback control system 310 (an embodiment of the feedback control system 210). The feedback control system 310 can include a feedback analyzer 312 and a controller circuit 318. In an example, the imaging signal may pass through an optical splitter before reaching the feedback analyzer 312. The feedback analyzer 312 may include a spectrometer that may generate one or more spectroscopic properties from the imaging data. The feedback analyzer 312 may recognize the target as a calculi target or anatomical target at or near the surgical site, or classify the target as one type of tissue or one type of calculi of distinct composition using the one or more spectroscopic properties. In some examples, the feedback analyzer 312 may calculate or estimate the fiber-target distance using the spectroscopic properties. The controller circuit 318 may generate a control signal to the laser source 332 to adjust a laser output setting, a control signal to the actuator 338 to adjust the position or orientation of the distal end 346 of an irrigation and/or suction channel 344 (e.g., the fiber-tissue distance, or an aiming angle), or a control signal to the irrigation and/or suction system 340 to adjust irrigation flow or suction flow, based on the structure, composition, or type of the target.

In some examples, the camera or imaging device 325 can include an imaging sensor (such as the imaging sensor 222) that can generate images or video frames of the target. The images or video frames of the surgical site can be transmitted to the feedback analyzer 312. Similar to the feedback analyzer 212, the feedback analyzer 312 (an embodiment of the feedback analyzer 212) can detect an indication of tissue whitening from the images or video frames of the surgical site. In an example, the tissue whitening may be determined based on a comparison of the intensity of the color components (e.g., RGB values) to threshold values (RGBm) for a pre-determined “whitened” color. In another example, the feedback analyzer 312 can perform frame-to-frame comparison of the video frames to determine a color intensity change rate towards white (RGB (255, 255, 255)) or the pre-determined “whitened” color. The feedback analyzer 312 can determine a degree of tissue whitening ΔRGB based on the difference between the intensity values (RGB values) of an image or video frame taken at time t (RGB) and the threshold values RGB_{TH}, or a rate of tissue whitening ΔRGB/Δt, as discussed above with reference to FIG. 2.

In some examples, the feedback analyzer 312 can detect tissue whitening (and determine the tissue whitening degree ΔRGB or the tissue whitening rate ΔRGB/Δt) from a portion of the images or video frames at or in a vicinity of a footprint of the aiming beam incident on the tissue at the surgical site, as described with reference to FIG. 4. Tissue whitening (including the degree and rate of tissue whitening) can be detected based on an increase in color intensity at or in a vicinity of the aiming beam footprint.

The detection of tissue whitening, including the degree and/or the rate of tissue whitening, may be used by the controller circuit 318 to regulate surgical site temperature, such as by adjusting operating parameters of one or more devices such as the laser source 332, the irrigation and/or suction system 340, or the irrigant treatment unit 342.

The irrigation and/or suction system 340 (an embodiment of the irrigation and/or suction system 240) can include an irrigation source and a suction source, each fluidly coupled to a working channel of the endoscope 301, such as an irrigation and/or suction channel 344. The irrigation and/or suction channel 344 can be a common, unified channel for conducting irrigation inflow and suction outflow at different times. Alternatively, in some examples, the irrigation and/or suction channel 344 can comprise two separate channels, such as an irrigation channel and a suction channel. The separate irrigation channel and the suction channel may be parallel to each other, or coaxially disposed with a common axis, such as in a nested configuration. The irrigation source may function to provide irrigation fluid (irrigant) to the irrigation and/or suction channel 344. The irrigation fluid may be gravity fed or pressurized. In an example, a pump may produce pressurized irrigation flow through the irrigation and/or suction channel 344 into the surgical site. The suction source may function to pull, suck, draw, aspirate, or otherwise move or remove fluid and unwanted matters from the surgical site to a receptacle. The suction source may perform the aforementioned functions by generating and applying vacuum, suction, or negative pressure to the irrigation and/or suction channel 344.

The feedback analyzer 312 of the feedback control system 310 can receive feedback information produced by one or more sensors, including, for example, the imaging sensor in the camera or imaging device 325, and a pressure sensor 224 configured to sense a surgical site pressure during the procedure. The pressure sensor 224 can be located at a distal end 336 of the optical pathway 334. Alternatively, the pressure sensor 224 may be located at other locations, such as a distal end 346 of an irrigation and/or suction channel 344. As described above, the feedback analyzer 312 can detect an indication of tissue whitening from the images or video frames of the surgical site, and determine the tissue whitening degree ΔRGB or the tissue whitening rate ΔRGB/Δt. In accordance with the degree and/or the rate of identified tissue whitening, the controller circuit 318 (an embodiment of the controller circuit 218) can automatically, or prompt the user to manually, adjust one or more system parameters to regulate the surgical site temperature to prevent or reduce the severity of laser-induced tissue thermal damage.

Various temperature control means can be used to regulate surgical site temperature during a procedure. In an example, the controller circuit 318 can generate a control signal to the laser source 332 to automatically adjust a laser output setting, including one or more laser irradiation parameters, in accordance with the degree of tissue whitening ΔRGB or the rate of tissue whitening ΔRGB/Δt. In an example, the laser output settings may be adjusted by altering pulse sequencing (i.e., changing the temporal distribution of laser pulses within a specified time) to produce a non-equilibrium, or more chaotic and unpredictable irrigation flow in which the bubbles strike a wide range of tissue areas rather than the single tissue area at which tissue whitening would be observed, as described above with reference to FIGS. 5B and 5D.

In addition or alternative to adjusting laser output settings, in some examples, in some examples, the controller circuit 318 can generate a control signal to an actuator 338 to adjust a position of a laser emitting end relative to the target at the surgical site. The actuator 338 can be coupled to a portion of the optical pathway 334, and can be in electrical communication with the controller circuit 318. In an example, the actuator 338 may be located at or near the distal end of the endoscope 301. The actuator 338 may include one or more of an electromagnetic element, an electrostatic element, a piezoelectric element, or other actuating element such as to actuate or otherwise permit longitudinal or rotational positioning of the distal end 336 of the optical pathway 334 with respect to the working channel or other longitudinal passage of the endoscope 301, or with respect to another reference location for which the endoscope 301 may serve as a frame of reference. In response to the identified tissue whitening, the controller circuit 318 can activate the actuator 338 to adjust the position or orientation of a distal end 336 of the optical pathway 334, such as adjusting the longitudinal position by moving the distal end 336 farther away from the surgical site (to increase the fiber-target distance), and/or adjusting the rotational position by steering the distal end 336 away from the surgical site (to increase the aiming angle).

In yet another example, the controller circuit 318 can generate a control signal to the irrigation and/or suction system 340 to automatically adjust one or more irrigation parameters, such as an irrigation flow or a suction flow. The irrigation flow or suction flow can help dissipate the heat generated during the procedure (e.g., laser treatment of tissue or calculi fragmentation). The irrigation flow or a suction flow may also assist in removal of fluid and unwanted matters (e.g., tissue debris or stone fragments), and keep the pressure of the surgical site under control, such as to maintain the pressure at substantially at a user-specified pressure level (e.g., the user-specified pressure with a tolerance such as ±5-10%). In response to the identified tissue whitening, the controller circuit 318 can control the irrigation and/or suction system 340 to automatically increase the irrigation flow into the surgical site to increase convective heat transfer, and/or increase the suction flow (or suction pressure) to withdraw the fluid away from the surgical site to improve heat dissipation and reduce the surgical site temperature. In some examples, the irrigation flow or the suction flow can be selectively activated or adjusted based on the surgical site pressure monitored via the pressure sensor 224, as described above with reference to FIG. 2.

In another example, the controller circuit 318 can generate a control signal to the irrigant treatment unit 342 to automatically adjust the temperature of the irrigant before being applied to the surgical site. The irrigant treatment unit 342 can include a cooling system (e.g., a radiator, or an in-line chiller) to cool the irrigant, or a fluid mixer to mix at least two irrigant of different temperatures. In response to the identified tissue whitening, the controller circuit 318 can control the irrigation and/or suction system 340 to automatically cool the irrigant via the cooling system or the fluid mixer. The irrigant/suction system 340 can then apply the cooled irrigate to the surgical site via the irrigation and/or suction channel 344 to improve convective heat transfer therein and effectively and efficiently reduce the surgical site temperature.

The controller circuit 318 can generate, or receive from a user, a temperature management plan that defines a prioritized order of two or more temperature control means as described above, including, for example, changing a laser output setting or one or more laser irradiation parameters, adjusting the position or orientation of the distal portion of the optical pathway (e.g., a laser fiber), activating or adjusting an irrigation flow into the surgical site and/or a suction flow away from the surgical site, or altering the temperature of the irrigant before being applied to the surgical site, among other means.

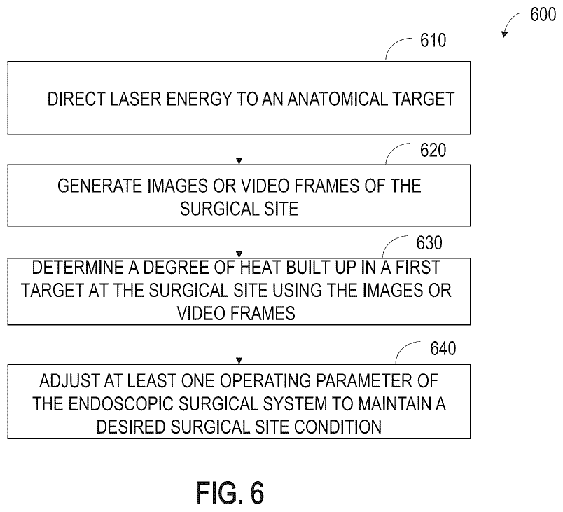

FIG. 6 is a flowchart illustrating a method 600 for controlling surgical site condition (such as surgical site temperature) during an endoscopic procedure for treating an anatomical target (e.g., soft tissue, hard tissue, cancerous tissue, or a calculi structure such as kidney or pancreobiliary or gallbladder stone). The method 600 may be implemented in and executed by the endoscopic surgical system 200, or the endoscopic laser lithotripsy system 300. Although the processes of the method 600 are drawn in one flowchart, they are not required to be performed in a particular order. In various examples, some of the processes may be performed in a different order than that illustrated herein.

At 610, laser energy (e.g., laser beams or a sequence of laser pulses) are delivered to an anatomical target. The laser energy may be generated by a laser source (such as the first laser source 106, the second laser source 116, or the laser source 332), and transmitted through an optical pathway (such as the first optical pathway 108, the second optical pathway 118, or the optical pathway 334). At 620, images or video frames of the surgical site taken at different times can be generated using an imaging sensor, such as the imaging sensor 222. At 630, the images or video frames may be analyzed, such as using the image analyzer circuit 214, and used to determine whether a degree of heat built up in a first target (e.g., tissue) at the surgical site exceeds a predetermined threshold. In an example, the determination of the degree of heat buildup can be based on whether tissue whitening (lightening of tissue color) can be detected in the images or video frames and the degree of the detected tissue whitening. In an example, images or video frames taken at different times can be compared to each other to identify a change in intensity of one or more color components (e.g., RGB values) at a registered location in the images or video frames. The registered location, which corresponds to a tissue site, can be specified by the user. In an example, the intensity of one or more color components can be compared to respective threshold values (RGBm) for a “white” color (RGB (255, 255, 255)) or a pre-determined “whitened” color (e.g., RGB (240, 240, 240)). If the intensity values (e.g., from 0 to 255) of one or more color components are close enough to the “white” color within a specified margin (e.g., RGB values within a range of (240-255, 240-255, 240-255), then a tissue whitening is deemed detected at 630. In some examples, a frame-to-frame comparison of the video frames, or a comparison of the consecutively captured images can be used to determine a color intensity change rate towards the “white” color (RGB (255, 255, 255)) or a pre-determined “whitened” color at the registered location in the images or video frames. A tissue whitening is deemed if the color intensity change rate exceeds a rate threshold. The tissue whitening thus detected indicates that the degree of heat built up in the tissue at the surgical site exceeds a threshold corresponding to RGBm or the threshold color intensity change rate.

In some examples, in addition to detecting the indication of tissue whitening, a degree of tissue whitening and/or a rate of tissue whitening can be determined at 630. The degree of tissue whitening can be determined based on a difference between the intensity values of the color components (e.g., RGB values) of an image or video frame taken at time t (RGBₜ) and the threshold values RGB_{TH} for the color components representing the “white” color or the pre-determined “whitened” color, that is, ΔRGB=RGBₜ−RGB_{TH}. A smaller ΔRGB value indicates a higher degree of tissue whitening, thus a higher temperature at the surgical site and a higher risk of tissue thermal damage. The rate of tissue whitening ΔRGB/Δt represents an amount of color intensity change towards the “white” or the pre-determined “whitened” color in a unit time. A higher tissue whitening rate ΔRGB/Δt indicates a faster heat buildup at the surgical site thus a higher risk of tissue thermal damage.

In an example, an aiming beam can be used to assist in identifying tissue whitening at a registered location on the images or video frames. The aiming beam, such as generated by the second laser source 234 or other light source, may be emitted when the target is illuminated. The aiming beam can have a distinct color (e.g., a green light in the range of approximately 520 nm, or a red light in the range of approximately 620 nm) to distinguish from the illumined background of the surgical site, as illustrated in FIG. 4. The aiming beam incident on the tissue at the surgical site, when falling within the field of view of the imaging sensor, can be captured by the imaging sensor. A footprint of the aiming beam can be identified from the images or video frames of the surgical site. When a colored (e.g., green) aiming beam is used, the aiming beam footprint can also be shown in green in the images or video frames. As described above with reference to FIG. 4, when the tissue becomes “whitened”, a colored (e.g., green or red) aiming beam incident on the “whitened” tissue can appear brighter in the image or video frames than on the “un-whitened” tissue. Tissue whitening can be detected from a portion of the images or video frames at or in a vicinity of the aiming beam footprint. A degree of tissue whitening ΔRGB, or a tissue whitening rate ΔRGB/Δt, can also be determined based on an increase in color intensity at or in a vicinity of the aiming beam footprint.

At 640, at least one operating parameter associated with the endoscopic surgical system may be adjusted based at least in part on the degree of heat built up in the first target at the surgical site, such as the tissue whitening detected from the images or video frames using the controller circuit 218 or the controller circuit 318. By adjusting the at least one operating parameter, a desired surgical site condition, such as a desired temperature at the surgical site, can be maintained during the procedure. Maintaining a desired surgical site condition (e.g., temperature) can help achieve or maintain a treatment effect (e.g., ablation or fragmentation of a calculi target) at the surgical site, while avoid potential tissue thermal damage due to laser-induced overheating at the surgical site. The adjustment of the at least one operating parameter can be carried out automatically by, for example, the controller circuit 218 or the controller circuit 318 electrically coupled to various devices of the endoscopic surgical system. Alternatively, the identified tissue whitening may be presented to a user, such as via the user interface device 250. The user may be alerted about temperature rise at the surgical site, and recommended to take proper preventive actions such as adjusting laser output or other system parameters.

Various temperature control means may be attempted, including, for example, changing a laser output setting or one or more laser irradiation parameters, adjusting the position or orientation of the distal portion of the optical pathway (e.g., a laser fiber), activating or adjusting an irrigation flow into the surgical site and/or a suction flow away from the surgical site, or altering the temperature of the irrigant before being applied to the surgical site, among other means, as described above with reference to FIGS. 2 and 3. In some examples, surgical site temperature can be controlled in accordance with a temperature management plan. The temperature management plan can include a prioritized order of two or more temperature control means described above. The temperature management plan can be programmed or modified by a user, such as via the user interface device 250. The order of the temperature control means can be determined based on the availability (e.g., irrigant cooling system), efficiency of temperature control, or potential adverse effects on the surgical site. In some examples, an aggressiveness of parameter adjustment (e.g., by adjusting the laser output setting, or irrigation and/or suction flow rate, or irrigant temperature control, etc.) can be determined based on the degree of tissue whitening ΔRGB, or the rate of tissue whitening ΔRGB/Δt. For example, if a higher degree of tissue whitening or a higher rate tissue whitening rate (i.e., a faster tissue whitening) is detected, the controller circuit 218 can provide more aggressive adjustments of one or more system parameters (e.g., more significant reduction in laser output, a higher irrigation flow rate and/or a higher suction flow rate, or more chilling of the irrigant before flowing into the surgical site) to bring the surgical site temperature under control.

FIG. 7A is a flowchart illustrating an example method of generating such a temperature management plan based on surgical site conditions including, for example, surgical site whitening and surgical site pressure. The temperature management plan can include a prioritized order of two or more temperature control means as described above. At 701, the tissue whitening identified at 630 can be evaluated against a tissue whitening criterion, such as the degree of tissue whitening ΔRGB falling below a threshold or the rate of tissue whitening exceeding a rate threshold. At 702, pressure can be sensed at the surgical site, such as using the pressure sensor 224. At 703, the measured surgical site pressure, P, can be compared to a pre-determine or user-specified upper pressure limit Pₘₐₓ, also referred to as a maximal allowable pressure. At 704, a temperature management plan can be determined based on the temperature check at 701 and the pressure check at 703. Availability (e.g., irrigant cooling system) and efficiency of temperature control means, or potential adverse effects on the surgical site, may also be considered to determine an individualized temperature management plan for the patient. The temperature management plan can include a prioritized order of two or more temperature control means as described above, including, for example, changing a laser output setting or one or more laser irradiation parameters, adjusting the position or orientation of the distal portion of the optical pathway (e.g., a laser fiber), activating or adjusting an irrigation flow into the surgical site and/or a suction flow away from the surgical site, or altering the temperature of the irrigant before being applied to the surgical site, among other means. In an example as shown in FIG. 7A, when the temperature trend or the prediction of future surgical site temperature satisfies the temperature adjustment criterion at 701, the prioritized order between adjusting the laser output setting and adjusting irrigation or suction flow can be based at least in part on whether the surgical site pressure, P, reaches a level substantially close to the upper pressure limit Pₘₐₓ at 705. If the surgical site pressure P is substantially below Pₘₐₓ (e.g., the difference between P and Pₘₐₓ exceeds a threshold), then the irrigation flow rate can be increased at 706. If the surgical site pressure P is substantially close to Pₘₐₓ (e.g., within a user-specified or pre-determined margin of Pmax, e.g., +10%), then the priority of temperature control means can be determined based on whether an option to increase suction flow is available at 707. If the suction flow is not available or not activated by the user, then the laser output setting can be adjusted, such as by decreasing laser output at 708. However, if at 707 the suction flow is available and activated by the user, then the suction flow rate can be increased at 709. Following the temperature control operations at any of steps 706, 708, and 709, other temperature control means may be attempted in an on-demand mode (e.g., activated by the user). The monitoring of surgical site temperature can be continued at 620.

FIG. 7B is a flowchart illustrating an example of a temperature management plan with prioritized means for controlling surgical site temperature, which can be an embodiment of step 640 of adjusting at least one operating parameter associated with the endoscopic surgical system to achieve or maintain substantially a desired temperature at the surgical site. At 710, the tissue whitening identified at 630 can be evaluated against a tissue whitening criterion, such as the degree of tissue whitening ΔRGB falling below a threshold or the rate of tissue whitening exceeding a rate threshold. If the criterion is not satisfied at 710, then the surgical site temperature is deemed normal and no parameter is to be adjusted, and the monitoring of surgical site temperature can be continued at 620. If the criterion is satisfied at 710, then an option for cooling the irrigant before flowing into the surgical site is provided at 720. If irrigant cooling option is available and selected (e.g., by the user), then at 722 irrigant can be cooled before reaching the surgical site, such as using a cooling system (e.g., a radiator or an in-line chiller included in the irrigant treatment unit 342), or by mixing at least two irrigant sources of different temperatures. The cooled irrigate can be applied to the surgical site to improve convective heat transfer therein. The monitoring of surgical site temperature can be continued at 620.

If the irrigant cooling option is not available or not selected at 720, then an option for using an irrigation and/or suction system (such as the irrigation and/or suction system 240 or the irrigation and/or suction system 340) is provided at 730. As discussed above with reference to FIGS. 2 and 3, the irrigation and/or suction system can provide an irrigation inflow to the surgical site, and/or a suction flow (outflow) of fluid out of the surgical site. In addition to assisting in removal of the tissue debris, stone fragments, and other unwanted matters during the procedure, the irrigation flow and suction flow also have a cooling effect on the tissue at or near the surgical site. If at 730 the irrigation and/or suction option is not available or not selected (e.g., by the user), then laser output settings can be adjusted at 732. For example, an average power of laser pulses can be reduced, such as by reducing one or more of a pulse width of a laser pulse, a peak power of a laser pulse, or a pulse frequency representing a number of laser pulses per unit time. Reducing the average power of laser pulses can decrease the laser-induced heating effect at or near the surgical site, thereby preventing tissue thermal damage and improving patient safety during the procedure. The amount (aggressiveness) of adjustment of the laser output settings, including one or more laser irradiation parameters, can be in accordance with the degree of tissue whitening or the rate of tissue whitening. In an example, the laser irradiation parameters to be adjusted at 732 can include a pulse sequencing parameter representing timings, or temporal distributions, of laser pulses within a specific time. As described above with reference to FIGS. SA and SC, a constant pulse sequencing of the laser may facilitate laser-induced vaporized bubbles travelling in an equilibrium state to a constant point of contact with the tissue wall. Under such equilibrium state, a predictable turbulent flow of bubbles toward the same tissue area can constantly strike the tissue area and cause heat buildup. As described above with reference to FIGS. 5B and 5D, adjusting the laser pulse sequencing can help redistribute the bubbles across a wide range of tissue areas, and prevent the bubbles from traveling in an equilibrium state and continuously striking the same area of tissue. The non-equilibrium state of flow may also promote collapses of vaporized bubbles and reduce the number and sizes of vaporized bubbles, thereby lowering the surgical site temperature and preventing tissue thermal damage.

In addition or alternative to adjusting the laser output settings including one or more laser irradiation parameters, in some examples, a position or orientation of a distal portion of an optical pathway relative to the anatomical target at the surgical site can be adjusted, such as via the actuator 338 to adjust the position or orientation of the distal end 336 of the optical pathway 334. The position or orientation can be adjusted to increase the fiber-target distance and/or to increase the aiming angle, thereby reducing the density of the laser energy incident on the surgical site and the laser-induced heat transferred into the surgical site. The monitoring of surgical site temperature can be continued at 620.

If the irrigation and/or suction option is available and selected at 730, then at 740 a pressure can be sensed at the surgical site, such as using the pressure sensor 224. Depending on the sensed pressure (P) at the surgical site, one or both of the irrigation flow or the suction flow may be selectively activated or adjusted to achieve temperature control at the surgical site temperature. At 750, the measured surgical site pressure is compared to a pre-determine or user-specified upper pressure limit Pₘₐₓ. If the measured surgical site pressure exceeds the Pₘₐₓ (P>Pₘₐₓ), then at 752 only the suction flow rate (but not the irrigation flow rate) is increased to lower the surgical site temperature. Additionally or alternatively, the irrigation flow rate may be reduced to reduce the pressure at the surgical site. As an increase in irrigation flow into the surgical site may introduce a positive pressure change at or near the surgical site, further increase in irrigation flow should be avoided to prevent further increase in surgical site pressure. If the measured surgical site pressure is lower than Pₘₐₓ, the measured surgical site pressure can be further compared to a pre-determine or user-specified a lower pressure limit Pₘᵢₙ at 760. If the measured surgical site pressure is within a range defined by Pₘᵢₙ and Pₘₐₓ (Pₘᵢₙ<P<Pₘₐₓ), then at 770 one or both of the irrigation flow rate or the suction flow rate can be increased to lower the surgical site temperature. However, if at 560 the measured surgical site pressure falls below the lower pressure limit Pₘᵢₙ (P<Pₘᵢₙ) then at 762 only the irrigation flow rate into the surgical site (but not the suction flow rate) is increased to lower the surgical site temperature, but avoid increasing the suction flow to prevent further decrease in surgical site pressure. Additionally or alternatively, the suction flow rate may be reduced to increase the pressure at the surgical site. As an increase in suction flow may introduce a negative pressure change at or near the surgical site, further increase in suction flow should be avoided to prevent further decrease in surgical site pressure. After the adjustment of irrigation or suction flow at 752, 762, or 770, the monitoring of surgical site temperature can be continued at 620.

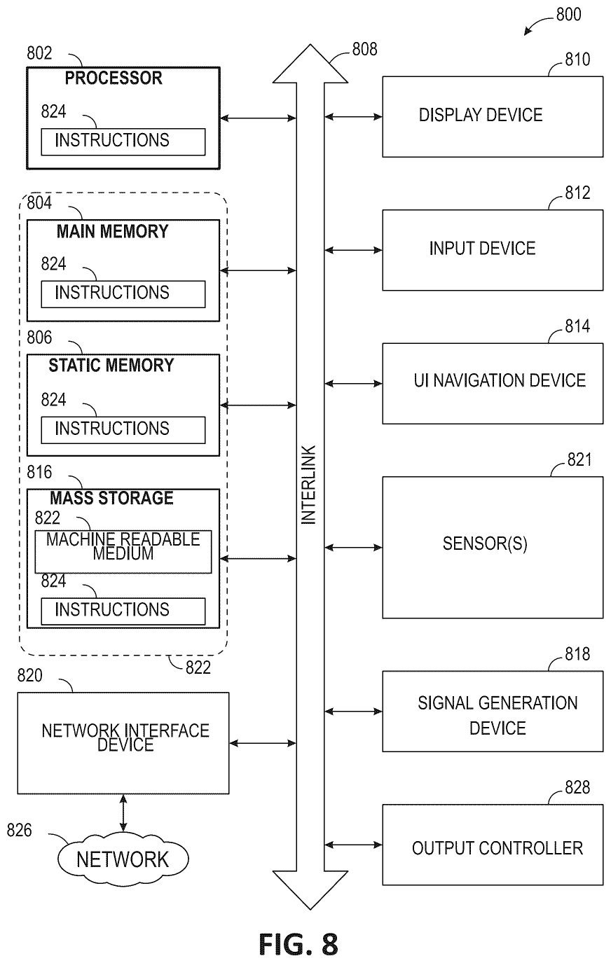

FIG. 8 illustrates generally a block diagram of an example machine 800 upon which any one or more of the techniques (e.g., methodologies) discussed herein may perform. Portions of this description may apply to the computing framework of various portions of the endoscopic surgical system 200 or the endoscopic surgical system 300.

In alternative embodiments, the machine 800 may operate as a standalone device or may be connected (e.g., networked) to other machines. In a networked deployment, the machine 800 may operate in the capacity of a server machine, a client machine, or both in server-client network environments. In an example, the machine 800 may act as a peer machine in peer-to-peer (P2P)(or other distributed) network environment. The machine 800 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 800 may include a hardware processor 802 (e.g., a central processing unit (CPU), a graphics processing unit (GPU), a hardware processor core, or any combination thereof), a main memory 804 and a static memory 806, some or all of which may communicate with each other via an interlink (e.g., bus) 808. The machine 800 may further include a display unit 810 (e.g., a raster display, vector display, holographic display, etc.), an alphanumeric input device 812 (e.g., a keyboard), and a user interface (UI) navigation device 814 (e.g., a mouse). In an example, the display unit 810, input device 812 and UI navigation device 814 may be a touch screen display. The machine 800 may additionally include a storage device (e.g., drive unit) 816, a signal generation device 818 (e.g., a speaker), a network interface device 820, and one or more sensors 821, such as a global positioning system (GPS) sensor, compass, accelerometer, or other sensors. The machine 800 may include an output controller 828, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 816 may include a machine readable medium 822 on which is stored one or more sets of data structures or instructions 824 (e.g., software) embodying or utilized by any one or more of the techniques or functions described herein. The instructions 824 may also reside, completely or at least partially, within the main memory 804, within static memory 806, or within the hardware processor 802 during execution thereof by the machine 800. In an example, one or any combination of the hardware processor 802, the main memory 804, the static memory 806, or the storage device 816 may constitute machine readable media.

While the machine-readable medium 822 is illustrated as a single medium, the term “machine readable medium” may include a single medium or multiple media (e.g., a centralized or distributed database, and/or associated caches and servers) configured to store the one or more instructions 824.

The term “machine readable medium” may include any medium that is capable of storing, encoding, or carrying instructions for execution by the machine 800 and that cause the machine 800 to perform any one or more of the techniques of the present disclosure, or that is capable of storing, encoding or carrying data structures used by or associated with such instructions. Non-limiting machine-readable medium examples may include solid-state memories, and optical and magnetic media. In an example, a massed machine-readable medium comprises a machine readable medium with a plurality of particles having invariant (e.g., rest) mass. Accordingly, massed machine-readable media are not transitory propagating signals. Specific examples of massed machine-readable media may include: non-volatile memory, such as semiconductor memory devices (e.g., Electrically Programmable Read-Only Memory (EPROM), Electrically Erasable Programmable Read-Only Memory (EPSOM)) and flash memory devices; magnetic disks, such as internal hard disks and removable disks; magneto-optical disks; and CD-ROM and DVD-ROM disks.

The instructions 824 may further be transmitted or received over a communication network 826 using a transmission medium via the network interface device 820 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as WiFi, IEEE 802.16 family of standards known as WiMax®), IEEE 802.15.4 family of standards, peer-to-peer (P2P) networks, among others. In an example, the network interface device 820 may include one or more physical jacks (e.g., Ethernet, coaxial, or phone jacks) or one or more antennas to connect to the communication network 826. In an example, the network interface device 820 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 800, and includes digital or analog communications signals or other intangible medium to facilitate communication of such software.

### Additional Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of“at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A endoscopic surgical system, comprising:
an endoscopic surgical device, controllably coupled to a medical instrument for delivering energy to a surgical site during a procedure;
an imaging sensor configured to generate images or video frames of at least a portion of the surgical site during the procedure; and
a controller circuit configured to:
analyze the generated images or video frames to determine whether a degree of heat built up in a first target at the surgical site exceeds a predetermined threshold; and
based on the determination, determine whether to adjust at least one operating parameter associated with the endoscopic surgical system so as to achieve or maintain a treatment effect of a second target at the surgical site while avoid damaging the first target during the procedure, the second target being different from the first target.

2. The endoscopic surgical system of claim 1, wherein the first target comprises tissue in a urinary system, the second target comprises a calculi target, and the medical instrument comprises at least one laser system for delivering laser energy to treat the calculi target at the surgical site.

3. The endoscopic surgical system of claim 1, wherein the controller circuit is further configured to:
detect, in the images or video frames, a change in intensity of one or more color components over time associated with the first target in the images or video frames; and
determine whether tissue whitening has occurred at the first target based on the detected change in intensity of the one or more color components.

4. The endoscopic surgical system of claim 1, wherein:
the endoscopic surgical device is configured to direct an aiming beam from a light source to the surgical site, the aiming beam having a characteristic color component; and
the controller circuit is further configured to identify a footprint of the aiming beam in the generated images or video frames, and determine the degree of heat built up in the first target based on an increase in intensity of the characteristic color component at a vicinity of the footprint of the aiming beam.

5. The endoscopic surgical system of claim 1, wherein the controller circuit is further configured to:
determine a rate of heat built up in the first target based on a comparison of the images or video frames taken at different times during the procedure; and
adjust the at least one operating parameter associated with the endoscopic surgical system in accordance with the determined degree or rate of tissue whitening.

6. The endoscopic surgical system of claim 2, wherein the at least one operating parameter to be adjusted includes a laser output setting of the at least one laser system,
the controller circuit is further configured to adjust the laser output setting so as to produce a non-equilibrium irrigation flow or to promote collapses of vaporized bubbles induced by the laser energy.

7. The endoscopic surgical system of claim 6, wherein the laser output setting to be adjusted comprises a pulse sequencing representing a temporal distribution of laser pulses within a specific time interval, the laser pulses being delivered to the second target in accordance with the adjusted pulse sequencing.

8. The endoscopic surgical system of claim 6, wherein to adjust the laser output setting, the controller circuit is further configured to prioritize adjustments of a pulse shape or a pulse sequencing over an adjustment of an average power of the laser pulses.

9. The endoscopic surgical system of claim 1, further comprising an irrigation and/or suction system configured to provide irrigant into, and suction of fluid from, the surgical site.

10. The endoscopic surgical system of claim 9, wherein the at least one operating parameter associated with the endoscopic surgical system comprises at least one of an irrigation flow or a suction flow associated with an irrigation system and a suction system, respectively.

11. The endoscopic surgical system of claim 10, further comprising a pressure sensor configured to sense a pressure at the surgical site during the procedure,
wherein the controller circuit is further configured to selectively increase the irrigation flow or the suction flow via the irrigation and/or suction system, including to:
increase the suction flow but not the irrigation flow when the sensed pressure exceeds an upper pressure limit;
increase one or both of the irrigation flow or the suction flow when the sensed pressure is within a range defined by the upper pressure limit and a lower pressure limit; and
increase the irrigation flow but not the suction flow when the sensed pressure falls below the lower pressure limit.

12. The endoscopic surgical system of claim 9, further comprising an irrigant treatment unit configured to alter a temperature of the irrigant,
wherein the controller circuit is further configured to generate a control signal to the irrigant treatment unit to adjust the temperature of the irrigant before reaching the surgical site upon determining that the degree of heat built up in the first target exceeds the predetermined threshold.

13. The endoscopic surgical system of claim 2, wherein the endoscopic surgical device includes an optical pathway with an adjustable distal portion, the optical pathway configured to direct the laser energy to the surgical site,
wherein the controller circuit is further configured to, upon determining that the degree of heat built up in the first target exceeds the predetermined threshold, generate a control signal to an actuator coupled to the optical pathway to adjust a position or orientation of the distal portion of the optical pathway relative to the surgical site.

14. The endoscopic surgical system of claim 1, wherein the at least one operating parameter associated with the endoscopic surgical system comprises at least one of:
temperature of an irrigant before being applied to the surgical site;
an irrigation flow rate;
a suction flow rate; or
a laser output setting of a laser system.

15. The endoscopic surgical system of claim 14, wherein the controller circuit is further configured to perform the adjustment with a bias toward one of the operating parameters based at least in part on at least one of the degree of heat built up in the first target or a pressure at the surgical site.

16. A method for controlling temperature at a surgical site of a patient during an endoscopic procedure using an endoscopic surgical system, the method comprising:
directing energy produced by a medical instrument to the surgical site;
generating images or video frames of at least a portion of the surgical site using an imaging sensor;
analyzing the generated images or video frames and determining whether a degree of heat built up in a first target at the surgical site exceeds a predetermined threshold; and
based on the determination, determining whether to adjust at least one operating parameter associated with the endoscopic surgical system so as to achieve or maintain a treatment effect of a second target at the surgical site while avoid damaging the first target during the procedure, the second target being different from the first target.

17. The method of claim 16, wherein the first target comprises tissue in a urinary system, the second target comprises a calculi target, and the energy produced by the medical instrument comprises laser energy produced by at least one laser system to treat the calculi target at the surgical site.

18. The method of claim 17, wherein the at least one operating parameter to be adjusted includes a pulse sequencing representing a temporal distribution of laser pulses within a specific time interval, the laser pulses being delivered to the surgical site in accordance with the adjusted pulse sequencing producing a non-equilibrium irrigation flow and promoting collapses of vaporized bubbles induced by the laser pulses.

19. The method of claim 18, wherein adjusting the pulse sequencing includes randomizing respective timings of the laser pulses within the specific time interval.

20. The method of claim 16, further comprising:
detecting, in the images or video frames, a change in intensity of one or more color components over time associated with the first target in the images or video frames; and
determining whether tissue whitening has occurred at the first target based at least in part on the detected change in intensity of the one or more color components.

21. The method of claim 16, further comprising:
directing an aiming beam from a light source to the surgical site, the aiming beam having a characteristic color component;
identifying a footprint of the aiming beam in the generated images or video frames; and
determine the degree of heat built up in the first target based at least in part on an increase in intensity of the characteristic color component at a vicinity of the footprint of the aiming beam.

22. The method of claim 16, further comprising:
determining a rate of heat built up in the first target based on a comparison of the images or video frames taken at different times during the procedure; and
adjusting the at least one operating parameter associated with the endoscopic surgical system in accordance with the determined degree or rate of tissue whitening.

23. The method of claim 16, wherein adjusting the at least one operating parameter associated with the endoscopic surgical system includes adjusting at least one of an irrigation flow of irrigant into the surgical site or a suction flow of fluid out of the surgical site.

24. The method of claim 16, wherein adjusting the at least one operating parameter includes adjusting, via an irrigant treatment unit coupled to an irrigation and/or suction system, a temperature of the irrigant before reaching the surgical site upon determining that the degree of heat built up in the first target exceeds the predetermined threshold.

25. The method of claim 16, wherein adjusting the at least one operating parameter includes, upon determining that the degree of heat built up in the first target exceeds the predetermined threshold, adjusting a position or orientation of a distal portion of an optical pathway relative to the surgical site, and directing the energy to the surgical site via the optical pathway.

26. The method of claim 16, comprising prioritizing adjustments of two or more operating parameters of the endocopic surgical system including:
a temperature of an irrigant before being applied to the surgical site;
an irrigation flow rate;
a suction flow rate; and
a laser output setting of a laser system.
